(19) United States
(12) Patent Application Publication
(10) Pub. No.: US 2010/0101643 A1
(43) Pub. Date: Apr. 29, 2010

(54) COMPOUND, PHOTOELECTRIC CONVERTER AND PHOTOELECTROCHEMICAL CELL

(75) Inventors: Toshiya Takahashi, Toyonaka-shi (JP); Mutsuko Higo, Toyonaka-shi (JP); Tetsuya Shintaku, Toyonaka-shi (JP); Tetsuo Kawata, Ibaraki-shi (JP); Akio Tanaka, Kobe-shi (JP); Kunihito Miyake, Tsukuba-shi (JP)

Correspondence Address: BIRCH STEWART KOLASCH & BIRCH, PO BOX 747, FALLS CHURCH, VA, 22040-0747, US

(73) Assignee: SUMITOMO CHEMICAL COMPANY, LIMITED, Tokyo (JP)

(21) Appl. No.: 12/593,539

(22) PCT Filed: Mar. 27, 2008
(86) PCT No.: PCT/JP2008/056644

§ 371 Date: Nov. 12, 2009

(30) Foreign Application Priority Data
Mar. 29, 2007 (JP) 2007-087281
Mar. 29, 2007 (JP) 2007-087291

(51) Int. Cl. H01L 31/0216; C07D 401/14; C07F 15/00

(52) U.S. Cl. 136/256; 546/256; 546/4

(57) ABSTRACT

A complex compound (I) obtained by coordinating a compound represented by the following formula (II), hereinafter abbreviated as compound (II), to a metal atom. In the formula, R¹, R² and R³ each independently represent a substituent represented by the following formula (III), formula (IV), formula (V) or formula (VI) and at least one of them is a substituent represented by the formula (III); a, b and c each independently represent an integer of 0 to 2 and a+b+c≧1; here, L represents a linking group represented by the following formula (VII) or formula (VIII); Ar represents an aryl group which may have a substituent; A represents an acidic group or a salt thereof; Y represents a halogen atom or a substituent; Q¹ and Q² each independently represent a hydrogen atom, an alkyl group having 1 to 20 carbon atoms, an alkoxy group having 1 to 20 carbon atoms, an aryl group having 6 to 20 carbon atoms or a cyano group; and p and q each represent an integer of 1 to 3.

### TECHNICAL FIELD

The present invention relates to a compound, a photosensitizing dye comprising the compound, a photoelectric converter comprising the dye, and a photoelectrochemical cell such as a solar cell comprising the photoelectric converter.

### BACKGROUND ART

In recent years, reduction in CO₂ emitted into the atmosphere has been required in order to prevent global warming. As an important means to decrease CO₂, for example, conversion to a solar system is proposed, where a photoelectrochemical cell such as a p-n junction-type, silicon-based solar cell is disposed on a house roof. However, the monocrystalline, polycrystalline and amorphous silicon used in the silicon-based photoelectrochemical cell has been expensive because, during the manufacturing processes thereof, high temperature and high vacuum conditions are necessary.

On the other hand, in Application Example A of National

Publication of International Patent Application No. Hei-7-500630 and J. Phys. Chem. B, 2003, 107, pp. 8981-8987, there is proposed a photoelectrochemical cell comprising a photoelectric converter which comprises semiconductor fine particles such as titanium dioxide, on the surface of which is adsorbed a photosensitizing dye which is easy to manufacture. Specifically, it is reported that the compounds represented by the following formula (1) and formula (2) show an excellent photoelectric conversion efficiency.

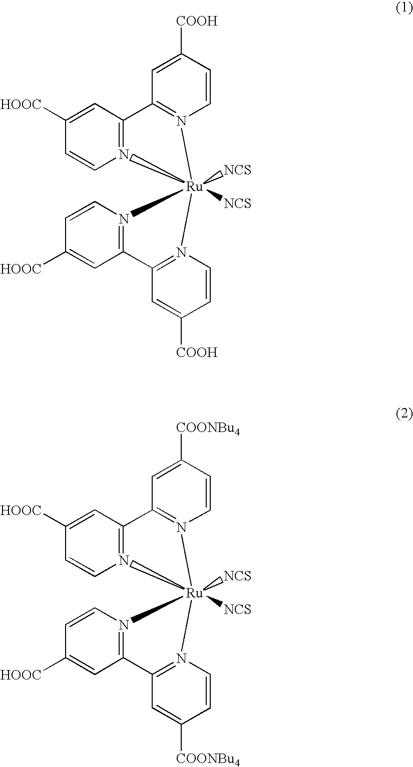

When the present inventors conducted a study on a photoelectrochemical cell comprising the photosensitizing dyes (1) and (2), it became clear that the photoelectric conversion efficiency in the visible light region to a long-wavelength region, especially in a long-wavelength region of 750 nm or longer, was not sufficient.

An object of the present invention is to provide a compound which provides a photoelectric converter having a high photoelectric conversion efficiency in a wide region from the visible light region to a long-wavelength region, a photosensitizing dye comprising the compound, a photoelectric converter comprising the dye and a photoelectrochemical cell comprising the converter.

### SUMMARY OF INVENTION

The present invention is a complex compound (I) wherein a ligand represented by the formula (II) and a bidendate ligand are coordinated to a metal atom; a photosensitizing dye comprising the complex compound (I); a photoelectric converter comprising the dye; and a photoelectrochemical cell comprising the converter:

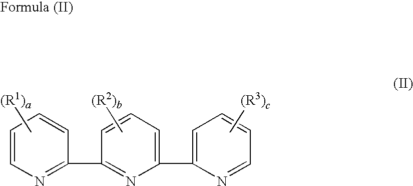

wherein, R¹, R² and R³ each independently represent a substituent represented by the following formula (III), formula (IV), formula (V) or formula (VI), and at least one of these is a substituent represented by the formula (III); a, b and c each independently represent 0 or an integer of 1 to 2 and a+b+c≧1:

-L-Ar-A (III)

-L-Ar—Y (IV)

-A (V)

—Y (VI)

wherein L represents a linking group represented by the following formula (VII) or formula (VIII); Ar represents an aryl group which may have a substituent; A represents an acidic group or a salt thereof; Q¹ and Q² represent each independently a hydrogen atom, an alkyl group having 1 to 20 carbon atoms, an alkoxy group having 1 to 20 carbon atoms, an aryl group having 6 to 20 carbon atoms or a cyano group; p and q each represent an integer of 1 to 3:

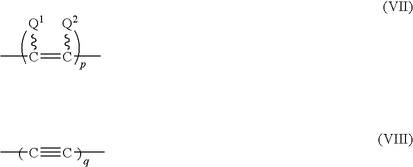

and Y represents at least one group selected from the group consisting of a hydrogen atom, an alkyl group having 1 to 20 carbon atoms, an alkoxy group having 1 to 20 carbon atoms, an alkoxyalkyl group having 2 to 20 carbon atoms, an aryloxy group having 6 to 20 carbon atoms, an arylalkyloxy group having 7 to 20 carbon atoms, an aryloxyalkyl group having 7 to 20 carbon atoms, an alkylthio group having 1 to 20 carbon atoms, an alkylthioalkyl group having 2 to 20 carbon atoms, an arylthio group having 6 to 20 carbon atoms, an arylalkylthio group having 7 to 20 carbon atoms, an arylthioalkyl group having 7 to 20 carbon atoms, an alkylsulfonyl group having 1 to 20 carbon atoms, an arylsulfonyl group having 6 to 20 carbon atoms, an amino group containing two of an alkyl group having 1 to 20 carbon atoms or an aryl group having 6 to 20 carbon atoms and a cyano group.]

The present invention is also a compound represented by the following formula (II′), abbreviated as compound (II′), and a method for manufacturing the same; a complex compound (I′) wherein compound (II′) is coordinated to a metal atom; a photosensitizing dye comprising the complex compound (I′); a photoelectric converter comprising the dye; and a photoelectrochemical cell comprising the converter: Formula (II′)

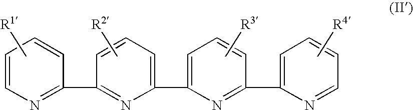

wherein, R¹′, R²′, R³′ and R⁴′ are each independent, at least one of R¹′ to R⁴′ is an acidic group or a salt thereof, at least one of them is represented by the formula (III′):

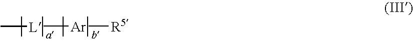

and at least one of them is represented by the formula (III') where a′=1; wherein a′ and b′ are each independent and represent an integer of 0 or 1; R¹′ to R⁵′ represent one group selected from the group consisting of an acidic group or a salt thereof, a hydrogen atom, an alkyl group having 1 to 20 carbon atoms, an alkoxy group having 1 to 20 carbon, an alkoxyalkyl group having 2 to 20 carbon atoms, an aryloxy group having 6 to 20 carbon atoms, an arylalkyloxy group having 7 to 20 carbon atoms, an aryloxyalkyl group having 7 to 20 carbon atoms, an ester group having 2 to 20 carbon atoms, an alkylthio group having 1 to 20 carbon atoms, an alkylthioalkyl group having 2 to 20 carbon atoms, an arylthio group having 6 to 20 carbon atoms, an arylalkylthio group having 7 to 20 carbon atoms, am arylthioalkyl group having 7 to 20 carbon atoms, an alkylsulfonyl group having 1 to 20 carbon atoms, an arylsulfonyl group having 6 to 20 carbon atoms, an amino group containing two of an alkyl group having 1 to 20 carbon atoms or an aryl groups having 6 to 20 carbon atoms and a cyano group; Ar represents an aryl group which may have a substituent; and L′ is a group represented by the following formula (IV′):

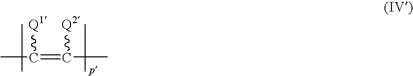

or the following formula (V′):

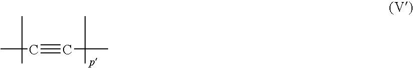

wherein, Q¹′ and Q²′ each independently represent a hydrogen atom, an alkyl group having 1 to 20 carbon atoms, an alkoxy group having 1 to 20 carbon atoms, an aryl group having 6 to 20 carbon atoms or a cyano group; and p′ is an integer of 1 to 3.

Further, the method for manufacturing compound (II′) of the present invention comprises the following processes (A) to (C) or processes (A) and (B):

### [Process (A)]:

a process wherein a compound represented by the formula (1′), hereinafter abbreviated as compound (1′):

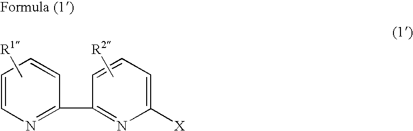

wherein, X represents a halogen atom;
and a compound represented by the formula (2′), hereinafter abbreviated as compound (2′):

(⁶′R)₃—Sn—Sn—(R⁶′)₃

wherein, R⁶′ represents an alkyl group having 1 to 4 carbon atoms;
are reacted to obtain a compound represented by the formula (3′), hereinafter abbreviated as compound (3′):

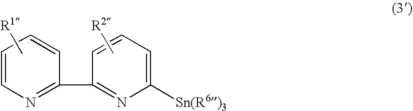

### [Process (B)]:

a process wherein the compound (3′) obtained in the process (A) and a compound represented by the formula (4′), hereinafter abbreviated as compound (4′):

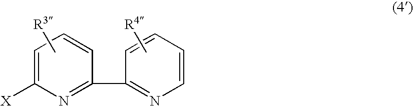

wherein, X represents a halogen atom;
are reacted in the presence of a metal catalyst to obtain a compound represented by the formula (5′), hereinafter abbreviated as compound (5′):

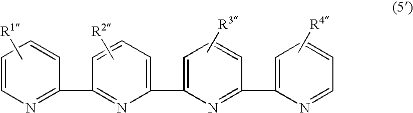

wherein, R¹″, R²″, R³″ and R⁴″ are each independent, at least one of R¹″ to R⁴″ is an acidic group to which a protecting group is introduced, and at least one of them is represented by the formula (VI′):

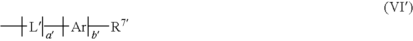

and at least one of them is a group represented by the formula (VI′) where a′=1; wherein, in the formula (VI′), a′, b′, Ar and L′ represent the same meanings as the definitions described in the formula (III′); R¹″ to R⁴″ and R⁷′ represent an acidic group to which a protecting group is introduced, a hydrogen atom or a substituent; and the substituent represents the same meaning as the definition described in the formula (III′).

### [Process (C)]:

a process wherein the compound (II′) is obtained by removing in a solvent the protecting group of compound (5′) obtained in the process (B).

### BRIEF DESCRIPTION OF DRAWINGS

FIG. 1 is a schematic cross-sectional view of a photoelectrochemical cell of the present invention.

### DESCRIPTION OF SYMBOLS

1 Substrate
2 Conductive layer
3 Layer of semiconductor particles
4 Photosensitizing dye
5 Electrolytic solution
6 Conductive layer
7 Substrate
8 Conductive substrate
9 Counter electrode (conductive substrate)
10 Sealant

### BEST MODES FOR CARRYING OUT THE INVENTION

Hereinafter, the present invention will be described in detail.

First, the compound (II) of the present invention, complex compound (I) wherein compound (II) is coordinated to a metal atom, and a method for manufacturing compound (II) will be described.

The metal atoms include Ti and Zr of Group 4; Fe, Ru and Os of Group 8; Co, Rh and Ir of Group 9; Ni, Pd and Pt of Group 10; Cu of Group 11; Zn of Group 12; and the like. Of these, preferable are the Group 8 metal atoms, more preferably Ru.

In the formula (II), R¹, R² and R³ each independently represent a substituent represented by the formula (III), formula (IV), formula (V) or formula (VI) and at least one of them is a substituent represented by the formula (III).

In the formula (III) and formula (IV), L represents a linking group expressed by the formula (VII) or formula (VIII). Q¹ and Q² each independently represent a hydrogen atom, an alkyl group having 1 to 20 carbon atoms, an alkoxy group having 1 to 20 carbon atoms, an aryl group having 6 to 20 carbon atoms or a cyano group, with a hydrogen atom being especially preferable; p and q represent an integer of 1 to 3, with p=1 or q=1 being preferable. In addition, the linking group expressed by the formula (VII) may be either an E-isomer or a Z-isomer, or may be a mixture of an E-isomer and a Z-isomer.

In the formula (III) or formula (IV), Ar represents an aryl group shown in the following.

Examples of Ar include the examples represented by the following formulae. In addition, the following exemplification shows that, of the hydrogen atoms substituted on the carbon atoms, two hydrogen atoms become the binding sites. Represented by * is a binding site with a substituent A or Y, and ** represents a binding site with one end of the linking group L. Further, another end of the linking group L is bound to a pyridine ring in the formula (II).

As Ar, a group represented by the formula (A-1) or (A-4) is preferable.

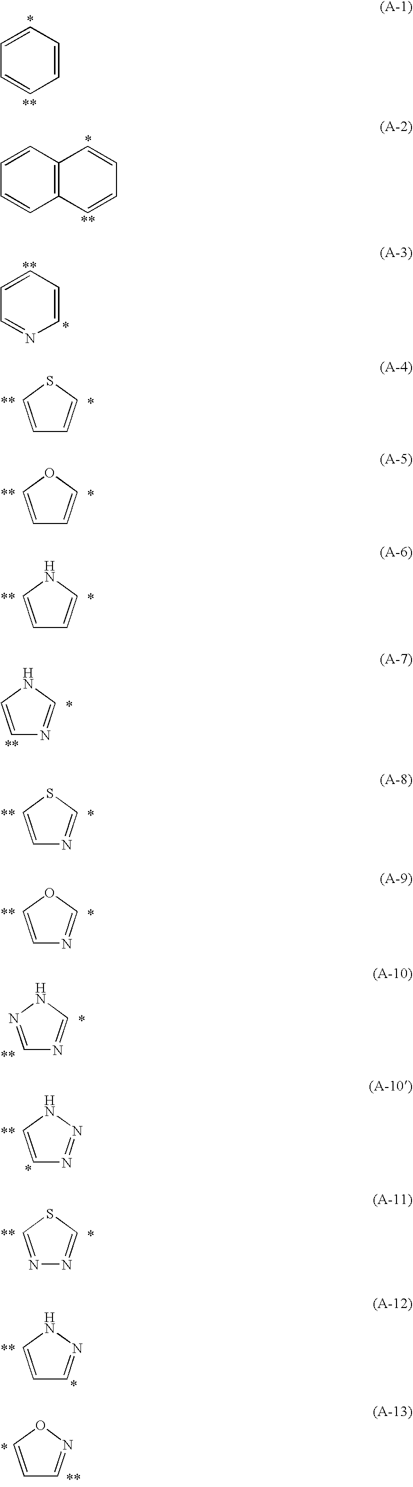

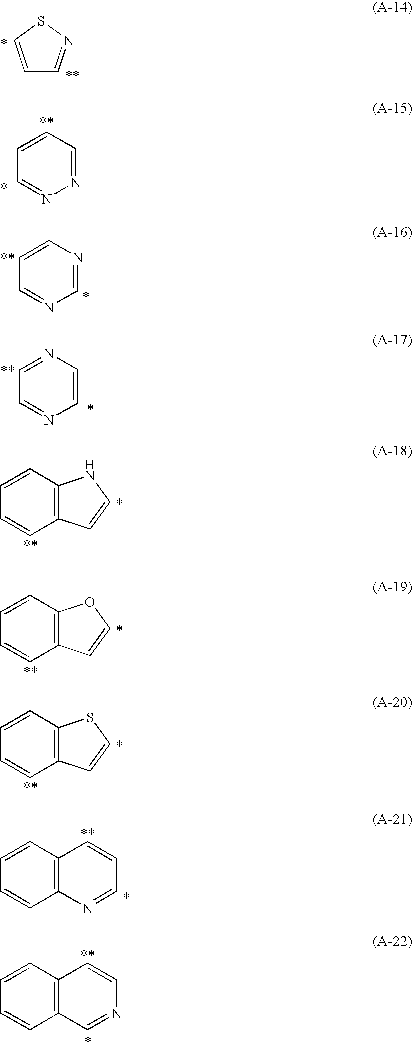

Hereinafter, substituents of Ar will be described. The substituents of Ar include an alkyl group having 1 to 20 carbon atoms, an alkoxy group having 1 to 20 carbon atoms, an alkoxyalkyl group having 2 to 20 carbon atoms, an aryloxy group having 6 to 20 carbon atoms, an arylalkyloxy group having 7 to 20 carbon atoms, an aryloxyalkyl group having 7 to 20 carbon atoms, an alkylthio group having 1 to 20 carbon atoms, an alkylthioalkyl group having 2 to 20 carbon atoms, an arylthio group having 6 to 20 carbon atoms, an arylalkylthio group having 7 to 20 carbon atoms, an arylthioalkyl group having 7 to 20 carbon atoms, an alkylsulfonyl group having 1 to 20 carbon atoms, an arylsulfonyl group having 6 to 20 carbon atoms, an amino group substituted by two alkyl groups having 1 to 20 carbon atoms or two aryl groups having 6 to 20 carbon atoms and a cyano group.

The alkyl group having 1 to 20 carbon atoms is preferably an alkyl group having 1 to 12 carbon atoms. The examples include linear alkyl groups such as a methyl group, an ethyl group, an n-propyl group, an n-butyl group, an n-hexyl group, an n-pentyl group, an n-octyl group, and an n-nonyl group; branched alkyl groups such as an i-propyl group, a t-butyl group, and a 2-ethylhexyl group; alicyclic alkyl groups such as a cyclopropyl group and a cyclohexyl group.

The aryl group has 6 to 20 carbon atoms and examples include a phenyl group and a naphthyl group.

Also, the carbon atom contained in an alkyl group or an aryl group may be substituted with an oxygen atom, a sulfur atom or a nitrogen atom.

The amino groups having two alkyl groups or two aryl groups include, for example, a dialkylamino group containing linear or branched alkyl groups such as a dimethylamino group, a diethylamino group, a dipropylamino group, a methylethylamino group, a methylhexylamino group, a methyloctylamino group; and a diarylamino group such as a diphenylamino group and a dinaphthylamino group.

In the formula (III) or formula (V), A represents an acidic group or a salt of an acidic group. The acidic groups include, for example, a carboxyl group, a sulfonic acid group (—SO₃H), a squaric acid group, a phosphoric acid (—PO₃H₂) group and a boric acid group (—B(OH)₂). Especially, the carboxyl group is preferable.

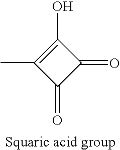

The salt of the acidic group includes a salt with an organic base. Specifically, there may be cited a tetraalkylammonium salt, an imidazolium salt, a pyridinium salt and the like.

In the formula (IV) or the formula (W), Y is a group selected from the group consisting of a hydrogen atom, an alkyl group having 1 to 20 carbon atoms, an alkoxy group having 1 to 20 carbon atoms, an alkoxyalkyl group having 2 to 20 carbon atoms, an aryloxy group having 6 to 20 carbon atoms, an arylalkyloxy group having 7 to 20 carbon atoms, an aryloxyalkyl group having 7 to 20 carbon atoms, an alkylthio group having 1 to 20 carbon atoms, an alkylthioalkyl group having 2 to 20 carbon atoms, an arylthio group having 6 to 20 carbon atoms, an arylalkylthio group having 7 to 20 carbon atoms, an arylthioalkyl group having 7 to 20 carbon atoms, an alkylsulfonyl group having 1 to 20 carbon atoms, an arylsulfonyl group having 6 to 20 carbon atoms, an amino group containing two of an alkyl group having 1 to 20 carbon atoms or an aryl group having 6 to 20 carbon atoms and a cyano group.

Here, the number of carbon atoms of the alkyl group is 1 to 20, preferably 1 to 12. The examples include linear alkyl groups such as a methyl group, an ethyl group, an n-propyl group, an n-butyl group, an n-hexyl group, an n-pentyl group, an n-octyl group, and an n-nonyl group; branched alkyl groups such as an i-propyl group, a t-butyl group, and a 2-ethylhexyl group; alicyclic alkyl groups such as a cyclopropyl group and a cyclohexyl group.

The aryl groups has 6 to 20 carbon atoms and examples include a phenyl group and a naphthyl group.

The carbon atom contained in an alkyl group or an aryl group may be substituted with an oxygen atom, a sulfur atom or a nitrogen atom.

The amino groups having two alkyl groups or two aryl groups include, for example, a dialkylamino group containing linear or branched alkyl groups such as a dimethylamino group, a diethylamino group, a dipropylamino group, a methylethylamino group, a methylhexylamino group, a methyloctylamino group; and a diarylamino group such as a diphenylamino group and a dinaphthylamino group.

In the formula (II), a, b and c each independently represent 0 or an integer of 1 to 2 and a+b+c≧1. Especially preferably, a+b+c is an integer of 1 to 3.

Of R¹, R² and R³, at least one contains a substituent represented by the formula (III), wherein preferable is a case where the linking group L is represented by the formula (VII), Q¹ and Q² are hydrogen atoms, p is 1, Ar is a thiophene ring which may have a substituent and A is a carboxyl group.

Hereinafter, a method for manufacturing compound (II) will be described.

When producing compound (II) of the present invention, depending on the kind of the acidic group A, there are sometimes cases where the Stille coupling reaction does not proceed. Therefore, it is possible to obtain the desired compound (II) by using a halogenated compound, the acidic group of which has been protected beforehand, and subjecting the same to stannylation and a coupling reaction to obtain compound (XVI), which is the hydrolyzed:

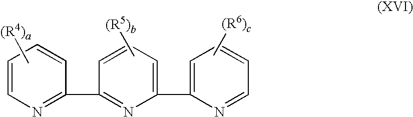

[In the formula (XVI), R⁴, R⁵ and R⁶ each independently represent a substituent represented by the formula (XII), formula (XIII), formula (XIV) or formula (XV) and at least one has a substituent represented by the formula (XII); and a, b and c each independently represent 0 or an integer of 1 to 2 and a+b+c≧1:

-L-Ar—B (XII)

-L-Ar—Y (XIII)

—B (XIV)

—Y (XV)

wherein, L, Ar and Y represent the same meaning as the L, Ar and Y in the formula (III), formula (IV), formula (V) and formula (VI), each of compound (II); B represents a substituent which is a protected form of the acidic group A in the formula (III) and (V).]

B can be obtained by, for example, esterifying the acidic group A with an alkyl group. The alkyl groups of the alkyl esters include alkyl groups having 1 to 10 carbon atoms, which may be substituted, and include a methyl group, an ethyl group, an n-propyl group, an isopropyl group, an n-butyl group, an isobutyl group and the like. Preferable are alkyl esters of carboxylic acids.

The aforementioned reactions will be described in more detail.

### [The First Step]:

A halogenated compound with its acidic group protected is stannylated. [In the following formula, R′—X represents a halogenated compound represented by the following formulae (IX) and (X). X represents the same meaning as the X in the halogenated compounds, (IX) and (X). R′″ represents an alkyl group.]

$${R}^{′} - X {}^{Tin reagent Metal catalyst} {R}^{′} - {Sn ( {R}^{′′′′} )}_{3}$$

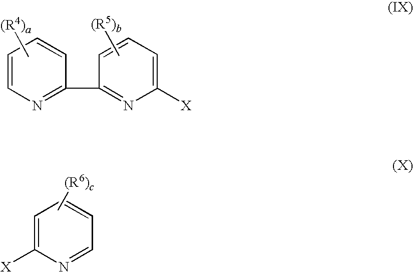

In the formula (IX) and formula (X), X represents a halogen atom and is preferably Br, Cl or I, especially preferably Br.

### [The Second Step]:

The tin compound obtained in the first step and a halogenated compound are subjected to a Stine coupling reaction. [In the following formula, R″—X represents the halogenated compounds represented by the formula (IX) and formula (X); X represents the same meaning as X in the halogenated compounds (IX) and (X); and R″—R′ represents the compound represented by the formula (XVI).]

$${R}^{″} - X + {R}^{′} - {Sn ( {R}^{′′′′} )}_{3} {\to }^{Metal catalyst} {R}^{″} - {R}^{′}$$

### [The Third Step]:

Next, the compound obtained is subjected to deprotection (hydrolysis reaction). [In the following formula, R′″ represents, among the compounds represented by the formula (XVI), the portions of formula (XII) and formula (XIV) other than the substituent B. B represents a substituent which is a protected form of the acidic group A. R′″-A represents compound (II) of the present invention.]

$${R}^{′′′} - B {}^{Acid or base} {R}^{′′′} - A$$

The stannylation methods using stannylating reagents include (1) a method to use an n-butyllithium/hexane solution and a halogenated alkyl tin, (2) a method to use alkyltin lithium, (3) a method to use a tin reagent represented by the following formula (XI) in the presence of a metal catalyst, and the like. The method (3) can be applied to many substituents. Especially, even though there are cases where stannylation of halogenated compounds does not proceed with methods (1) and (2), when the substituent R⁵ of the halogenated compound (IX) and the substituent R⁶ of the halogenated compound (X) are the substituents represented by the formula (XII) and formula (XIV), the reaction proceeds with the method (3) to obtain the compound (XVI).

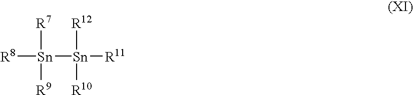

In the stannylating reagent (XI), R⁷ to R¹² each independently represent an alkyl group having 1 to 6 carbon atoms and include, for example, methyl, ethyl, n-propyl, isopropyl, n-butyl, isobutyl, s-butyl, tert-butyl, n-pentyl, 1-ethylpropyl, n-hexyl, isohexyl and 4-methylpentyl. Each of R⁷ to R¹² may be different or the same. Especially preferably, R⁷ to R¹² are all methyl groups or n-butyl groups. The amount of the stannylating reagent to be used is, relative to 1 mole of the halogenated compound, usually 1 to 50 equivalent times, preferably 1 to 5 equivalent times.

Hereinafter, the reaction of the first step will be described in more detail.

The metal catalysts used in the reaction include tetrakis(triphenylphosphine)palladium(0), dichlorobis(triphenylphosphine)palladium(II), bis[1,2-bis(diphenylphosphino)ethane]palladium(0), bis[o-phenylenebis(diethylphosphino)ethane(diphenylphosphino)palladium(0), bis(acetonitrile)dichloropalladium(II) and the like. Preferable among these are tetrakis(triphenylphosphine)palladium(0) and dichlorobis(triphenylphosphine)palladium(II).

Each may be used independently or in a combination. In addition, the reaction may be carried out in a heterogeneous system with the metal catalyst supported on a carrier such as a resin which is not soluble in the reaction solvent. In the reaction of the present invention, the amount of the metal catalyst to be used is, relative to 1 mole of the halogenated compound, at least 0.00001 equivalent times and at most 5 equivalent times, preferably at least 0.00001 equivalent times and at most 1 equivalent times.

The reaction is preferably carried out in a solvent.

As the solvent, there is no particular limitation as long as it does not interfere with the reaction and dissolves the starting material to some extent. Examples include aliphatic hydrocarbons such as hexane and heptane;

aromatic hydrocarbons such as benzene, toluene and xylene;

ethers such as diethyl ether, diisopropyl ether, 1,2-dimethoxyethane, tetrahydrofuran, dioxane and diethylene glycol dimethyl ethers;

nitriles such as acetonitrile, propionitrile and isobutyronitrile;

amides such as formamide, N,N-dimethylformamide, N,N-dimethylacetamide, N-methylpyrrolidone, N-methylpyrrolidinone and hexamethylphosphorotriamide.

The reaction temperature depends on the structure of the halogenated compound but is usually 0 to 200° C., preferably 50 to 150° C. The reaction time varies depending mainly on the reaction temperature, reaction raw material, reagent, additive or solvent used but is usually 5 minutes to 5 days, more preferably 15 minutes to 24 hours. When the reaction rate is slow, the reaction yield may be improved by elongating the reaction time further until the halogenated compound disappears. In addition, in order to prevent deactivation of the catalyst by oxygen during the reaction, the reaction is preferably carried out under an inert gas atmosphere. For example, inert gas such as nitrogen gas and argon gas may be mentioned. Further, the reaction pressure is not particularly limited but is usually carried out under atmospheric pressure. In the manufacturing method of the present invention, the order of charging the halogenated compound, metal catalyst and reaction solvent is not particularly limited; as an example, there may be mentioned a method whereby the halogenated compound, tin reagent and metal catalyst are mixed in the organic solvent. The obtained tin compound may, if necessary, be purified by providing such means as distillation, recrystallization, various chromatographies and the like.

When the first step reaction is carried out using compound (IX), reaction products represented by the following formula (A) are obtained and, when carried out using compound (X), reaction products represented by the following formula (B) are obtained.

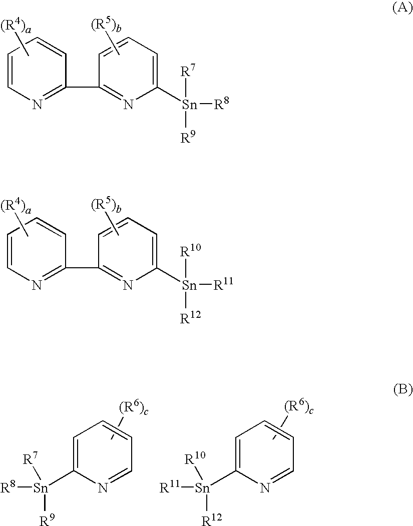

Next, the second step reaction will be described. As for the halogenated compound to be used in the Stille coupling reaction, when tin compound (A) was obtained by carrying out a stannylation reaction using compound (IX) in the first step, halogenated compound (X) may be reacted to obtain compound (XVI). Further, the same result will be obtained when a stannylation reaction is carried out using compound (X) to obtain tin compound (B), which is then reacted with halogenated compound (IX). The amount of the halogenated compound is, relative to 1 mole of the tin compound, usually at least 0.1 equivalent times and at most 1.0 equivalent times, preferably at least 0.7 equivalent times and at most 1.0 equivalent times. Furthermore, when tin compound (B) is used and reacted with following halogenated compound (XVII), compound (XVI) where R⁴═R⁶ can be obtained. In that case, the amount of halogenated compound (XVII) to be used is, relative to tin compound (X), usually at least 0.1 equivalent times and at most 0.5 equivalent times, preferably at least 0.3 equivalent times and at most 0.5 equivalent times, X represents a halogen atom and is preferably Br, Cl or I. An especially preferable halogenated compound is one with X═Br.

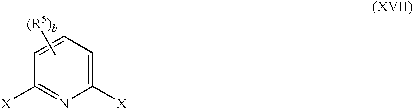

The metal catalyst and reaction solvent may be used by selecting from those exemplified in the first step and may be the same as or different from those used in the first step. The amount of the metal catalyst to be used is, relative to the halogenated compound, at least 0.00001 equivalent times and at most 1.0 equivalent times, preferably at least 0.00001 equivalent times and at most 0.2 equivalent times. The reaction temperature depends on the structure of the halogenated compound but is usually 0 to 200° C., preferably 50 to 150° C. The reaction time varies depending mainly on the reaction temperature, reaction raw material, reagent, additive or solvent used but is usually 5 minutes to 5 days, preferably 15 minutes to 24 hours. In addition, in order to prevent deactivation of the catalyst by oxygen during the reaction, the reaction is preferably carried out under an inert gas atmosphere.

For example, inert gas such as nitrogen gas and argon gas may be mentioned. Further, the reaction pressure is not particularly limited but is usually carried out under atmospheric pressure. In addition, there is no particular limitation on the order of charging the halogenated compound, metal catalyst and reaction solvent. As an example, there may be mentioned a method whereby the halogenated compound, tin reagent and metal catalyst are mixed in the organic solvent. Meanwhile, when the reaction rate is slow, the reaction yield may be improved by elongating the reaction time further until the time the halogenated compound disappears or by adding additional metal catalyst or tin compound.

The obtained compound (XVI) may, if necessary, be purified by providing such means as distillation, recrystallization, various chromatographies and the like.

Then, the third step reaction will be described. The hydrolysis reaction conducted here may be carried out by using either an acid or a base, but when the reaction is carried out using a base, it is possible to hydrolyze in a short period and under a mild condition. The bases used in the present invention include inorganic bases such as hydroxides or carbonate salts of alkali metals or alkaline earth metals, and oxides of alkaline earth metals. The alkali metal hydroxides include potassium hydroxide, sodium hydroxide and the like, and the alkali metal carbonates include potassium carbonate, sodium carbonate and the like. Among these, especially preferable are the alkali metal hydroxides. The amount of these bases to be used is, relative to compound (XVI), usually 1 to 50 equivalent times, preferably 1 to 5 equivalent times. In addition, there may be used two or more kinds of bases.

In the reaction, a solvent is usually used and the reaction is carried out preferably in an organic solvent.

The organic solvent is not particularly limited as long as it dissolves the starting material to some extent. The examples include halogenated hydrocarbons such as dichloromethane, 1,2-dichloroethane and chloroform;

esters such as ethyl acetate and butyl acetate;

ethers such as diethyl ether, diisopropyl ether, 1,2-dimethoxyethane, tetrahydrofuran, dioxane and diethylene glycol dimethyl ether;

nitriles such as acetonitrile, propionitrile and isobutyronitrile;

ketones such as acetone and methyl ethyl ketone;

alcohols such as methanol, ethanol and isopropyl alcohol;

amides such as formamide, N,N-dimethylformamide, N,N-dimethylacetamide, N-methylpyrrolidone, N-methylpyrrolidinone and hexamethylphosphorotriamide. Among these, alcohols such as methanol and ethanol are preferable because they dissolve the substrate, desired material and base to some extent. In addition, the organic solvent may be used alone or water may be added thereto in order to dissolve the inorganic base.

The reaction temperature can be set in a relatively wide range but is usually set in a range of 5 to 150° C., preferably in a range of 5 to 100° C. The reaction time is not particularly limited and the end point of the reaction is determined as the time when the raw material has disappeared. It is usually in a range of 5 minutes to 24 hours. In addition, there is no particular limitation on the order of addition of the raw materials. As an example, there may be mentioned a method whereby compound (XVI) and a base are mixed in the aforementioned solvent. The reaction may be carried out either under inert gas such as nitrogen gas and argon gas, or under air. The reaction pressure is not particularly limited but is usually conducted under atmospheric pressure.

In the present invention, when the reaction liquid after the hydrolysis is separated into two layers, an organic layer and an aqueous layer, the product is extracted with an organic solvent and, thereafter, the extraction solvent is concentrated and the desired compound can be obtained by crystallization and filtration. When the reaction liquid does not separate into an organic layer and an aqueous layer, the reaction liquid is concentrated to dryness and is neutralized with an acid. The acid used in neutralization is not particularly limited but hydrochloric acid and sulfuric acid are often used. The concentration thereof is not particularly limited but it is usually preferable to use an aqueous solution of 50% by weight or less. The amount of the acid to be used is preferably not less than an amount equivalent to a base which remained unreacted at the time of hydrolysis.

In order to complete neutralization, the pH is preferably 7 or lower. But in many cases, the desired material precipitates under an acidic condition and, thus, it is desirable to add an acid until the product precipitates.

When crystals precipitate during neutralization, the desired material can be obtained by filtration-separation and washing. If crystals do not precipitate, the liquid may be concentrated to dryness as it is. The product obtained can be purified by applying means of recrystallization, various chromatography and the like. In addition, the product may be used for synthesis of the complex compound (I) without purification.

Examples of compound (II) include compounds (II-1) to (II-73) represented by the following formula, Table 1-1 and Table 1-2. In Table 1-1 and Table 1-2, there are described the binding positions of the pyridine rings and substituents of (R¹)ₐ, (R²)_{b} and (R³)_{c}. In addition, in each pyridine ring, the nitrogen atom is situated at a position of 1, 1′, or 1″. In Table 1-1 and Table 1-2, III-1 to III-16 are substituents represented by the formula (III) and, in Table 1′, Ar, L, p and A which constitute the substituents are described. In addition, IV-1 in Table 1-1 and Table 1-2 is a substituent represented by the formula (IV) and, in Table 1″, Ar, L, p and Y which constitute the substituent is described.

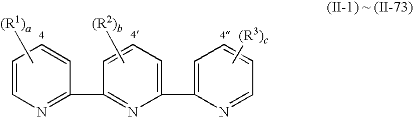

TABLE 1-1

| | R¹ | | | R² | | | R³ | | |
|---|---|---|---|---|---|---|---|---|---|
| Compound | a | Position | Substituent | b | Position | Substituent | c | Position | Substituent |
| II-1 | 1 | 4 | H | 1 | 4′ | III-1 | 1 | 4″ | H |
| II-2 | 1 | 4 | H | 1 | 4′ | III-1 | 1 | 4″ | III-1 |
| II-3 | 1 | 4 | —CH₃ | 1 | 4′ | —CH₃ | 1 | 4″ | III-1 |
| II-4 | 1 | 4 | III-1 | 1 | 4′ | —CH₃ | 1 | 4″ | III-1 |
| II-5 | 1 | 4 | III-1 | 1 | 4′ | III-1 | 1 | 4″ | III-1 |
| II-6 | 1 | 4 | H | 1 | 4′ | III-4 | 1 | 4″ | H |
| II-7 | 1 | 4 | H | 1 | 4′ | III-4 | 1 | 4″ | III-4 |
| II-8 | 1 | 4 | —CH₃ | 1 | 4′ | —CH₃ | 1 | 4″ | III-4 |
| II-9 | 1 | 4 | III-4 | 1 | 4′ | —CH₃ | 1 | 4″ | III-4 |
| II-10 | 1 | 4 | III-4 | 1 | 4′ | III-4 | 1 | 4″ | III-4 |
| II-11 | 1 | 4 | H | 1 | 4′ | III-5 | 1 | 4″ | H |
| II-12 | 1 | 4 | H | 1 | 4′ | III-5 | 1 | 4″ | III-5 |
| II-13 | 1 | 4 | —CH₃ | 1 | 4′ | —CH₃ | 1 | 4″ | III-5 |
| II-14 | 1 | 4 | III-5 | 1 | 4′ | —CH₃ | 1 | 4″ | III-5 |
| II-15 | 1 | 4 | III-5 | 1 | 4′ | III-5 | 1 | 4″ | III-5 |
| II-16 | 1 | 4 | H | 1 | 4′ | III-9 | 1 | 4″ | H |
| II-17 | 1 | 4 | —CH₃ | 1 | 4′ | III-9 | 1 | 4″ | —CH₃ |
| II-18 | 1 | 4 | —OCH₃ | 1 | 4′ | III-9 | 1 | 4″ | —OCH₃ |
| II-19 | 1 | 4 | —C₁₀H₂₁ | 1 | 4′ | III-9 | 1 | 4″ | —C₁₀H₂₁ |
| II-20 | 1 | 4 | H | 1 | 4′ | III-9 | 1 | 4″ | III-9 |
| II-21 | 1 | 4 | —CH₃ | 1 | 4′ | III-9 | 1 | 4″ | III-9 |
| II-22 | 1 | 4 | —OCH₃ | 1 | 4′ | III-9 | 1 | 4″ | III-9 |
| II-23 | 1 | 4 | —C₁₀H₂₁ | 1 | 4′ | III-9 | 1 | 4″ | III-9 |
| II-24 | 1 | 4 | H | 1 | 4′ | H | 1 | 4″ | III-9 |
| II-25 | 1 | 4 | —CH₃ | 1 | 4′ | —CH₃ | 1 | 4″ | III-9 |
| II-26 | 1 | 4 | —OCH₃ | 1 | 4′ | —OCH₃ | 1 | 4″ | III-9 |
| II-27 | 1 | 4 | —C₁₀H₂₁ | 1 | 4′ | —C₁₀H₂₁ | 1 | 4″ | III-9 |
| II-28 | 1 | 4 | —COOH | 1 | 4′ | H | 1 | 4″ | III-9 |
| II-29 | 1 | 4 | III-9 | 1 | 4′ | H | 1 | 4″ | III-9 |
| II-30 | 1 | 4 | III-9 | 1 | 4′ | —CH₃ | 1 | 4″ | III-9 |
| II-31 | 1 | 4 | III-9 | 1 | 4′ | —OCH₃ | 1 | 4″ | III-9 |
| II-32 | 1 | 4 | III-9 | 1 | 4′ | —C₁₀H₂₁ | 1 | 4″ | III-9 |
| II-33 | 1 | 4 | III-9 | 1 | 4′ | —COOH | 1 | 4″ | III-9 |
| II-34 | 1 | 4 | III-9 | 1 | 4′ | III-9 | 1 | 4″ | III-9 |
| II-35 | 1 | 4 | H | 1 | 4′ | III-10 | 1 | 4″ | H |
| II-36 | 1 | 4 | H | 1 | 4′ | III-10 | 1 | 4″ | III-10 |

TABLE 1-2

| | R¹ | | | R² | | | R³ | | |
|---|---|---|---|---|---|---|---|---|---|
| Compound | a | Position | Substituent | b | Position | Substituent | c | Position | Substituent |
| II-37 | 1 | 4 | —CH₃ | 1 | 4′ | —CH₃ | 1 | 4″ | III-10 |
| II-38 | 1 | 4 | III-10 | 1 | 4′ | —CH₃ | 1 | 4″ | III-10 |
| II-39 | 1 | 4 | H | 1 | 4′ | III-11 | 1 | 4″ | H |
| II-40 | 1 | 4 | H | 1 | 4′ | III-11 | 1 | 4″ | III-11 |
| II-41 | 1 | 4 | —CH₃ | 1 | 4′ | —CH₃ | 1 | 4″ | III-11 |
| II-42 | 1 | 4 | III-11 | 1 | 4′ | —CH₃ | 1 | 4″ | III-11 |
| II-43 | 1 | 4 | H | 1 | 4′ | III-12 | 1 | 4″ | H |
| II-44 | 1 | 4 | —CH₃ | 1 | 4′ | III-12 | 1 | 4″ | —CH₃ |
| II-45 | 1 | 4 | —OCH₃ | 1 | 4′ | III-12 | 1 | 4″ | —OCH₃ |
| II-46 | 1 | 4 | —C₁₀H₂₁ | 1 | 4′ | III-12 | 1 | 4″ | —C₁₀H₂₁ |
| II-47 | 1 | 4 | H | 1 | 4′ | III-12 | 1 | 4″ | III-12 |
| II-48 | 1 | 4 | —CH₃ | 1 | 4′ | III-12 | 1 | 4″ | III-12 |
| II-49 | 1 | 4 | —OCH₃ | 1 | 4′ | III-12 | 1 | 4″ | III-12 |
| II-50 | 1 | 4 | —C₁₀H₂₁ | 1 | 4′ | III-12 | 1 | 4″ | III-12 |
| II-51 | 1 | 4 | H | 1 | 4′ | H | 1 | 4″ | III-12 |
| II-52 | 1 | 4 | —CH₃ | 1 | 4′ | —CH₃ | 1 | 4″ | III-12 |
| II-53 | 1 | 4 | —OCH₃ | 1 | 4′ | —OCH₃ | 1 | 4″ | III-12 |
| II-54 | 1 | 4 | —C₁₀H₂₁ | 1 | 4′ | —C₁₀H₂₁ | 1 | 4″ | III-12 |
| II-55 | 1 | 4 | —COOH | 1 | 4′ | H | 1 | 4″ | III-12 |
| II-56 | 1 | 4 | III-12 | 1 | 4′ | H | 1 | 4″ | III-12 |
| II-57 | 1 | 4 | III-12 | 1 | 4′ | —CH₃ | 1 | 4″ | III-12 |
| II-58 | 1 | 4 | III-12 | 1 | 4′ | —OCH₃ | 1 | 4″ | III-12 |
| II-59 | 1 | 4 | III-12 | 1 | 4′ | —C₁₀H₂₁ | 1 | 4″ | III-12 |
| II-60 | 1 | 4 | III-12 | 1 | 4′ | —COOH | 1 | 4″ | III-12 |
| II-61 | 1 | 4 | III-12 | 1 | 4′ | III-12 | 1 | 4″ | III-12 |
| II-62 | 1 | 4 | H | 1 | 4′ | III-13 | 1 | 4″ | H |
| II-63 | 1 | 4 | H | 1 | 4′ | III-13 | 1 | 4″ | III-13 |
| II-64 | 1 | 4 | —CH₃ | 1 | 4′ | —CH₃ | 1 | 4″ | III-13 |
| II-65 | 1 | 4 | III-13 | 1 | 4′ | —CH₃ | 1 | 4″ | III-13 |
| II-66 | 1 | 4 | III-13 | 1 | 4′ | III-13 | 1 | 4″ | III-13 |
| II-67 | 1 | 4 | H | 1 | 4′ | III-16 | 1 | 4″ | H |
| II-68 | 1 | 4 | H | 1 | 4′ | III-16 | 1 | 4″ | III-16 |
| II-69 | 1 | 4 | —CH₃ | 1 | 4′ | —CH₃ | 1 | 4″ | III-16 |
| II-70 | 1 | 4 | III-16 | 1 | 4′ | —CH₃ | 1 | 4″ | III-16 |
| II-71 | 1 | 4 | III-16 | 1 | 4′ | III-16 | 1 | 4″ | III-16 |
| II-72 | 1 | 4 | IV-1 | 1 | 4′ | H | 1 | 4″ | III-9 |
| II-73 | 1 | 4 | IV-1 | 1 | 4′ | III-9 | 1 | 4″ | H |

TABLE 1′

| Substituent | Ar | L | p | A |
|---|---|---|---|---|
| III-1 | A-1 | —(CH═CH)— | 1 | —COOH |
| III-2 | A-1 | —(CH═CH)— | 1 | —SO3H |
| III-3 | A-1 | —(CH═CH)— | 1 | —PO3H2 |
| III-4 | A-1 | —(CH═CH)— | 1 | —COOTBA |
| III-5 | A-1 | —(C≡C)— | 1 | —COOH |
| III-6 | A-1 | —(C≡C)— | 1 | —SO3H |
| III-7 | A-1 | —(C≡C)— | 1 | —PO3H2 |
| III-8 | A-1 | —(C≡C)— | 1 | —COOTBA |
| III-9 | A-4 | —(CH═CH)— | 1 | —COOH |
| III-10 | A-4 | —(CH═CH)— | 1 | —SO3H |
| III-11 | A-4 | —(CH═CH)— | 1 | —PO3H2 |
| III-12 | A-4 | —(CH═CH)— | 1 | —COOTBA |
| III-13 | A-4 | —(C≡C)— | 1 | —COOH |
| III-14 | A-4 | —(C≡C)— | 1 | —SO3H |
| III-15 | A-4 | —(C≡C)— | 1 | —PO3H2 |
| III-16 | A-4 | —(C≡C)— | 1 | —COOTBA |

TBA = tetra-n-butylammonium salt

TABLE 1″

| Substituent | Ar | L | p | Y |
|---|---|---|---|---|
| IV-1 | A-1 | —(CH═CH)— | 1 | H |

Complex compound (I) of the present invention is obtained by having a compound represented by the aforementioned formula (II) coordinated to a metal atom.

In addition, complex compound (I) of the present invention comprises a metal atom M as the central atom and a compound represented by the formula (II) as one of the ligands.

There may be other ligands coordinated than the compound represented by the formula (II). Other ligands contained in complex compound (I) include, for example, isothiocyanate (—N═C═S, hereinafter may sometimes be referred to as NCS), thiocyanate (—S—C≡N, hereinafter may sometimes be referred to as SCN), diketonate, chloro, bromo, iodo, cyano and a hydroxyl group, with NCS or SCN being preferable. The complex compound may exist accompanied by counter anions such as halogen anions, in a form with the charge neutralized.

In the following, a method for manufacturing the complex compound (I) is described with the case of Ru, used as the metal atom, as an example.

There may be cited a method whereby an Ru reagent is dissolved in N,N-dimethylformamide or an alcoholic solvent and to the solution are mixed compound (II) at about 40 to 180° C. and, if necessary, a salt which provides an auxiliary ligand. From the resultant reaction solution, the complex compound is obtained by purification by recrystallization, chromatography or the like.

Here, as the Ru reagent is used a bivalent or trivalent Ru reagent. Specifically, these may be exemplified by RuCl₃, [RuCl₂(p-cymene)]₂ and RuCl₂(DMSO)₄. Specific examples of complex compound (I) include compounds (I-1) to (I-152) represented by the following formula and Table 2-1 to Table 2-4:

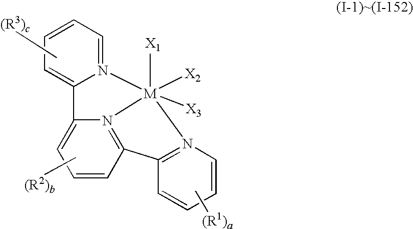

TABLE 2-1

| Complex | | Ligand | |
|---|---|---|---|
| compound | Metal atom M | Compound (II) | X1 = X2 = X3 |
| I-1 | Ru | II-1 | —NCS |
| I-2 | Ru | II-2 | —NCS |
| I-3 | Ru | II-3 | —NCS |
| I-4 | Ru | II-4 | —NCS |
| I-5 | Ru | II-5 | —NCS |
| I-6 | Ru | II-6 | —NCS |
| I-7 | Ru | II-7 | —NCS |
| I-8 | Ru | II-8 | —NCS |
| I-9 | Ru | II-9 | —NCS |
| I-10 | Ru | II-10 | —NCS |
| I-11 | Ru | II-11 | —NCS |
| I-12 | Ru | II-12 | —NCS |
| I-13 | Ru | II-13 | —NCS |
| I-14 | Ru | II-14 | —NCS |
| I-15 | Ru | II-15 | —NCS |
| I-16 | Ru | II-16 | —NCS |
| I-17 | Ru | II-17 | —NCS |
| I-18 | Ru | II-18 | —NCS |
| I-19 | Ru | II-19 | —NCS |
| I-20 | Ru | II-20 | —NCS |
| I-21 | Ru | II-21 | —NCS |
| I-22 | Ru | II-22 | —NCS |
| I-23 | Ru | II-23 | —NCS |
| I-24 | Ru | II-24 | —NCS |
| I-25 | Ru | II-25 | —NCS |
| I-26 | Ru | II-26 | —NCS |
| I-27 | Ru | II-27 | —NCS |
| I-28 | Ru | II-28 | —NCS |
| I-29 | Ru | II-29 | —NCS |
| I-30 | Ru | II-30 | —NCS |
| I-31 | Ru | II-31 | —NCS |
| I-32 | Ru | II-32 | —NCS |
| I-33 | Ru | II-33 | —NCS |
| I-34 | Ru | II-34 | —NCS |
| I-35 | Ru | II-35 | —NCS |
| I-36 | Ru | II-36 | —NCS |
| I-37 | Ru | II-37 | —NCS |
| I-38 | Ru | II-38 | —NCS |

TABLE 2-2

| Complex | | Ligand | |
|---|---|---|---|
| compound | Metal atom M | Compound (II) | X1 = X2 = X3 |
| I-39 | Ru | II-39 | —NCS |
| I-40 | Ru | II-40 | —NCS |
| I-41 | Ru | II-41 | —NCS |
| I-42 | Ru | II-42 | —NCS |
| I-43 | Ru | II-43 | —NCS |
| I-44 | Ru | II-44 | —NCS |
| I-45 | Ru | II-45 | —NCS |
| I-46 | Ru | II-46 | —NCS |
| I-47 | Ru | II-47 | —NCS |
| I-48 | Ru | II-48 | —NCS |
| I-49 | Ru | II-49 | —NCS |
| I-50 | Ru | II-50 | —NCS |
| I-51 | Ru | II-51 | —NCS |
| I-52 | Ru | II-52 | —NCS |
| I-53 | Ru | II-53 | —NCS |
| I-54 | Ru | II-54 | —NCS |
| I-55 | Ru | II-55 | —NCS |
| I-56 | Ru | II-56 | —NCS |
| I-57 | Ru | II-57 | —NCS |
| I-58 | Ru | II-58 | —NCS |
| I-59 | Ru | II-59 | —NCS |
| I-60 | Ru | II-60 | —NCS |
| I-61 | Ru | II-61 | —NCS |
| I-62 | Ru | II-62 | —NCS |
| I-63 | Ru | II-63 | —NCS |
| I-64 | Ru | II-64 | —NCS |
| I-65 | Ru | II-65 | —NCS |
| I-66 | Ru | II-66 | —NCS |
| I-67 | Ru | II-67 | —NCS |
| I-68 | Ru | II-68 | —NCS |
| I-69 | Ru | II-69 | —NCS |
| I-70 | Ru | II-70 | —NCS |
| I-71 | Ru | II-71 | —NCS |
| I-72 | Ru | II-72 | —NCS |
| I-73 | Ru | II-73 | —NCS |
| I-74 | Ru | II-16 | —SCN |
| I-75 | Ru | II-18 | —SCN |
| I-76 | Ru | II-19 | —SCN |

TABLE 2-3

| Complex | | Ligand | |
|---|---|---|---|
| compound | Metal atom M | Compound (II) | X1 = X2 = X3 |
| I-77 | Ru | II-20 | —SCN |
| I-78 | Ru | II-22 | —SCN |
| I-79 | Ru | II-23 | —SCN |
| I-80 | Ru | II-24 | —SCN |
| I-81 | Ru | II-25 | —SCN |
| I-82 | Ru | II-27 | —SCN |
| I-83 | Ru | II-29 | —SCN |
| I-84 | Ru | II-30 | —SCN |
| I-85 | Ru | II-32 | —SCN |
| I-86 | Ru | II-34 | —SCN |
| I-87 | Ru | II-45 | —SCN |
| I-88 | Ru | II-47 | —SCN |
| I-89 | Ru | II-48 | —SCN |
| I-90 | Ru | II-49 | —SCN |
| I-91 | Ru | II-51 | —SCN |
| I-92 | Ru | II-52 | —SCN |
| I-93 | Ru | II-53 | —SCN |
| I-94 | Ru | II-55 | —SCN |
| I-95 | Ru | II-56 | —SCN |
| I-96 | Ru | II-58 | —SCN |
| I-97 | Ru | II-60 | —SCN |
| I-98 | Ru | II-61 | —SCN |
| I-99 | Ru | II-63 | —SCN |
| I-100 | Ru | II-64 | —SCN |
| I-101 | Ru | II-65 | —SCN |
| I-102 | Ru | II-66 | —SCN |
| I-103 | Ru | II-67 | —SCN |
| I-104 | Ru | II-68 | —SCN |
| I-105 | Ru | II-69 | —SCN |
| I-106 | Ru | II-70 | —SCN |
| I-107 | Ru | II-71 | —SCN |
| I-108 | Ru | II-72 | —SCN |
| I-109 | Ru | II-73 | —SCN |
| I-110 | Ru | II-16 | —Cl |
| I-111 | Ru | II-18 | —Cl |
| I-112 | Ru | II-19 | —Cl |
| I-113 | Ru | II-20 | —Cl |
| I-114 | Ru | II-22 | —Cl |

TABLE 2-4

| Complex | | Ligand | X1 = X2 = |
|---|---|---|---|
| compound | Metal atom M | Compound (II) | X3 |
| I-115 | Ru | II-23 | —Cl |
| I-116 | Ru | II-24 | —Cl |
| I-117 | Ru | II-25 | —Cl |
| I-118 | Ru | II-27 | —Cl |
| I-119 | Ru | II-29 | —Cl |
| I-120 | Ru | II-30 | —Cl |
| I-121 | Ru | II-31 | —Cl |
| I-122 | Ru | II-32 | —Cl |
| I-123 | Ru | II-34 | —Cl |
| I-124 | Ru | II-45 | —Cl |
| I-125 | Ru | II-47 | —Cl |
| I-126 | Ru | II-48 | —Cl |
| I-127 | Ru | II-49 | —Cl |
| I-128 | Ru | II-51 | —Cl |
| I-129 | Ru | II-52 | —Cl |
| I-130 | Ru | II-53 | —Cl |
| I-131 | Ru | II-55 | —Cl |
| I-132 | Ru | II-56 | —Cl |
| I-133 | Ru | II-58 | —Cl |
| I-134 | Ru | II-60 | —Cl |
| I-135 | Ru | II-61 | —Cl |
| I-136 | Ru | II-63 | —Cl |
| I-137 | Ru | II-64 | —Cl |
| I-138 | Ru | II-65 | —Cl |
| I-139 | Ru | II-66 | —Cl |
| I-140 | Ru | II-67 | —Cl |
| I-141 | Ru | II-68 | —Cl |
| I-142 | Ru | II-69 | —Cl |
| I-143 | Ru | II-70 | —Cl |
| I-144 | Ru | II-71 | —Cl |
| I-145 | Ru | II-72 | —Cl |
| I-146 | Ru | II-73 | —Cl |
| I-147 | Os | II-16 | —NCS |
| I-148 | Os | II-25 | —NCS |
| I-149 | Os | II-30 | —NCS |
| I-150 | Fe | II-16 | —NCS |
| I-151 | Fe | II-25 | —NCS |
| I-152 | Fe | II-30 | —NCS |

Next, there will be described another embodiment of the present invention, which relates to a complex compound (I′) comprising a metal atom and a compound represented by the formula (II′), compound (II′), a method for producing the same, a photosensitizing dye comprising the complex compound, a photoelectric converter comprising the dye, and a photoelectrochemical cell such as a solar cell comprising the photoelectric converter.

The metal atoms include Ti and Zr of Group 4; Fe, Ru and Os of Group 8; Co, Rh and Ir of Group 9; Ni, Pd and Pt of Group 10; Cu of Group 11; Zn of Group 12 and the like. Of these, preferable are Group 8 metal atoms, more preferably Ru.

In the formula (I′) and formula (II′), R¹′, R²′, R³′ and R⁴′ are each independent, at least one of R¹″ to R⁴′ comprises an acidic group or a salt thereof, at least one of them is represented by the following formula (III′):

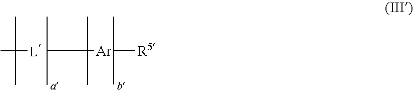

and at least one of them is represented by the formula (III′) where a′=1; a′ and b′ are each independent and represent an integer of 0 or 1. In complex compound (I′) and compound (II′), the number of the acidic groups or their salts is preferably 2 or more, more preferably 3 or more.

The acidic groups include a carboxyl group, a sulfonic acid group (—SO₃H), a squaric acid group, a phosphoric acid group (—PO₃H₂), a boric acid group (—B(OH)₂) and the like. Among these, the carboxyl group is preferable, especially from a viewpoint of synthetic simplicity.

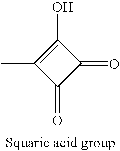

The salts include a salt with an organic base and, for example, there may be mentioned a tetraalkylammonium salt, an imidazolium salt and a pyridinium salt.

R¹′ to R⁵′ represent a group selected from the group consisting of an acidic group or a salt thereof, a hydrogen atom, an alkyl group having 1 to 20 carbon atoms, an alkoxy group having 1 to 20 carbon atoms, an alkoxyalkyl group having 2 to 20 carbon atoms, an aryloxy group having 6 to 20 carbon atoms, an arylalkyloxy group having 7 to 20 carbon atoms, an aryloxyalkyl group having 7 to 20 carbon atoms, an an arylthio group having 6 to 20 carbon atoms, an arylalkylthio group having 7 to 20 carbon atoms, an arylthioalkyl group having 7 to 20 carbon atoms, an alkylsulfonyl group having 1 to 20 carbon atoms, an arylsulfonyl group having 6 to 20 carbon atoms, an amino group containing two of an alkyl group having 1 to 20 carbon atoms or an aryl group having 6 to 20 carbon atoms and a cyano group.

The number of carbon atoms of the alkyl group is 1 to 20, preferably 1 to 12. The examples include linear alkyl groups such as a methyl group, an ethyl group, an n-propyl group, an n-butyl group, an n-hexyl group, an n-pentyl group, an n-octyl group, and an n-nonyl group; branched alkyl groups such as an i-propyl group, a t-butyl group, and a 2-ethylhexyl group; alicyclic alkyl groups such as a cyclopropyl group and a cyclohexyl group.

The number of carbon atoms of the alkoxy group is 1 to 20, preferably 1 to 12. The examples include a methoxy group, an ethoxy group, an n-propoxy group, an i-propoxy group, an n-butoxy group, a t-butoxy group and a decyloxy group.

The number of carbon atoms of the aryl group is 6 to 20. The examples include a phenyl group and a naphthyl group, which may have a substituent. The ester group contains 1 to 20 carbon atoms, preferably 1 to 5. Specific examples include a methyl ester group, an ethyl ester group, an n-propyl ester group, an n-butyl ester group, a t-butyl ester group and the like. Among these, preferable are the methyl ester group and ethyl ester group which are easy to synthesize economically.

The carbon atom contained in the alkyl or aryl group may be substituted by an oxygen atom, a sulfur atom or a nitrogen atom.

The amino group having two alkyl groups or two aryl groups include, for example, a dialkylamino group containing linear or branched alkyl groups such as a dimethylamino group, a diethyl amino group, a dipropylamino group, a methylethylamino group, a methylhexylamino group, a methyloctylamino group; and a diarylamino group such as a diphenylamino group and a dinaphthylamino group.

In the formula (III′), L′ is a group represented by the following formula (IV′):

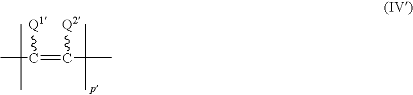

or by the following formula (V′):

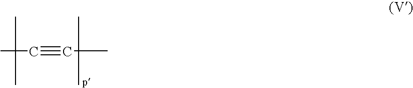

(wherein, Q¹′ and Q²′ each independently represent a hydrogen atom, an alkyl group having 1 to 20 carbon atoms, an alkoxy group having 1 to 20 carbon atoms, an aryl group having 6 to 20 carbon atoms or a cyano group; p represents an integer of 1 to 3.)

In the formula (IV′) or formula (V′), p′ represents an integer of 1 to 3, with p′=1 being preferable. In the formula (IV′), the structure may be either an E-isomer or a Z-isomer, or a mixture of the E-isomer and Z-isomer. Examples of Ar described in the formula (III′) include the above-described (A-1) to (A-22) but Ar is not limited to these. In the formula (III′), the signs * and ** in the examples show the binding positions, with * showing the position where Ar binds with R⁵′ described in the formula (III′). In L′, either of the unsaturated aliphatic hydrocarbon atoms is bound to the pyridine ring in the formula (II′) and another is bound to the binding position ** of Ar. Ar is preferably the group represented by the formula (A-1) or (A-4).

Examples of the substituents of Ar include a hydroxyl group, an alkyl group having 1 to 20 carbon atoms, an alkoxy group having 1 to 20 carbon atoms, an aryloxy group having 6 to 20 carbon atoms, a dialkylamino group having 2 to 20 carbon atoms and a diarylamino group having 12 to 20 carbon atoms. The alkyl groups include linear alkyl groups such as a methyl group, an ethyl group, an n-propyl group, an n-butyl group, an n-hexyl group, an n-pentyl group, an n-octyl group and an n-nonyl group; branched alkyl groups such as an i-propyl group, a t-butyl group, and a 2-ethylhexyl group; alicyclic alkyl groups such as a cyclopropyl group and a cyclohexyl group. The aryl groups include a phenyl group, a naphthyl group and the like.

The alkyl group has 1 to 20 carbon atoms, preferably 1 to 12 carbon atoms. Examples include linear alkyl groups such as a methyl group, an ethyl group, an n-propyl group, an n-butyl group, an n-hexyl group, an n-pentyl group, an n-octyl group, and an n-nonyl group; branched alkyl groups such as an i-propyl group, a t-butyl group, and a 2-ethylhexyl group; alicyclic alkyl groups such as a cyclopropyl group and a cyclohexyl group.

Specific examples of the alkoxy group include, for example, a methoxy group, an ethoxy group, an n-propoxy group, an i-propoxy group, an n-butoxy group, a t-butoxy group and a decyloxy group.

The aryl group has 6 to 20 carbon atoms, with examples including a phenyl group and a naphthyl group.

In addition, the carbon atom contained in the alkyl group or aryl group may be substituted by an oxygen atom, a sulfur atom or a nitrogen atom.

The amino group having two alkyl groups or two aryl groups include, for example, a dialkylamino group containing linear or branched alkyl groups such as a dimethylamino group, a diethylamino group, a dipropylamino group, a methylethylamino group, a methylhexylamino group, a methyloctylamino group; and a diarylamino group such as a diphenylamino group and a dinaphthylamino group.

Hereinafter, the process (A), process (B) and process (C) will be described in detail.

[Process (A)]

Process (A) is a process wherein compound (1′) is reacted with compound (2′) to obtain compound (3′).

When an acidic group is contained in compound (1′) used in process (A), it is preferable to have a protecting group introduced beforehand. For example, when the acidic group is a carboxyl group, it is mentioned that a protecting group is introduced therein beforehand to provide derivatives such as a methyl ester, an ethyl ester, an n-propyl ester, an n-butyl ester and the like.

From a standpoint of economic efficiency and synthetic ease, a methyl ester and an ethyl ester are preferable.

When the acidic group is a phosphoric acid group, it may be mentioned that protecting groups such as a methyl group, an ethyl group, an n-propyl group and an n-butyl group are introduced beforehand, with a methyl group and an ethyl group being preferable from a standpoint of economic efficiency.

The reaction solvents to be used include ethylene glycol dimethyl ether (hereinafter, abbreviated as DME), ethylene glycol diethyl ether, ethylene glycol butyl ether, tetrahydrofuran (hereinafter, abbreviated as THF) and the like, with DME being preferable from a standpoint of reactivity and economic efficiency.

The solvent is used, relative to 1 g of compound (1′), usually in an amount of 0.5 ml to 500 ml, preferably 0.7 ml to 400 ml, more preferably 1.0 ml to 350 ml.

The reaction temperature is usually 50° C. to 100° C., preferably 60° C. to 90° C., more preferably 65° C. to 85° C.

Compound (2′) includes hexamethylditin, hexabutylditin and the like, with hexamethylditin being preferable from a standpoint of reactivity. These compounds are commercially available and can be used as received or may be purified before use by, for example, distillation under reduced pressure.

The amount of compound (2′) to be used is, relative to 1 mole of compound (1′), usually 1.5 moles to 6 moles, preferably 2 moles to 5 moles, more preferably 2.5 moles to 4.5 moles.

The metal catalysts used include Ni, Pd, Pt and the like of Group 10, with Pd being preferable from a standpoint of reactivity. The Pd metal catalysts include Pd(PPh₃)₄ (PPh₃ represents triphenylphosphine) and Pd(PPh₃)₂Cl₂, with Pd(PPh₃)₄ being preferable from a standpoint of economic efficiency and easiness in handling.

The amount of the metal catalyst to be used is, relative to 1 mmole of compound (1), 20 micromoles to 100 micromoles, preferably 23 micromoles to 90 micromoles, more preferably 25 micromoles to 80 micromoles.

In process (A), the method of charging is not particularly limited but, from a standpoint of safety and operability, it is preferable to charge the solvent, compound (1′) and metal catalyst, and, thereafter, to add compound (2′), followed by heating.

The reaction time varies depending on the reagent used and the reaction temperature but is usually 0.5 hour to 10 hours, preferably 1 hour to 8 hours, more preferably 1.5 hours to 7 hours.

The degree of progress of the reaction can be confirmed by LC (liquid chromatography).

After completion of the reaction, there is partially observed a compound from which the protecting group is removed but the reaction mixture may be used in the next process without separation and purification of the reaction mixture. Alternatively, after cooling to room temperature, it is possible to separate and purify the reaction mixture by a usual aftertreatment.

As a purification method, for example, the reaction mixture is allowed to cool to room temperature and, thereafter, the solvent is distilled off by concentration under reduced pressure. An ether solvent (for example, diethyl ether) is added and the mixture is allowed to stand still or is stirred. As a solvent to be used, diethyl ether is especially preferable.

The time required for standing or stirring varies depending on the solvent used and temperature, but it is desirable to let the mixture stand still usually for 1 hour to 48 hours, preferably for 2 hours to 36 hours, more preferably for 3 hours to 25 hours.

The temperature at which the mixture is allowed to stand still or is stirred is usually −5° C. to 20° C., preferably −2° C. to 15° C., more preferably 0° C. to 10° C.

Thereafter, insoluble matter is removed by filtration. By concentrating the obtained filtrate under reduced pressure, compound (3′) can be purified.

[Process (B)]

Process (B) is a process wherein compound (3′) and compound (4′) are reacted in the presence of a metal catalyst to obtain compound (5′).

In compound (4′), X represents a halogen atom. From a standpoint of reactivity, an iodine atom, a bromine atom and a chlorine atom are preferable, with the bromine atom being especially preferable from a standpoint of yield. When an acidic group is contained in compound (4′), it is preferable to have a protecting group introduced beforehand. For example, when the acidic group is a carboxyl group, it is mentioned that a protecting group is introduced therein to provide derivatives such as a methyl ester, an ethyl ester, an n-propyl ester, an n-butyl ester and the like. From a standpoint of economic efficiency and synthetic ease, a methyl ester and an ethyl ester are especially preferable. When the acidic group is a phosphoric acid group, it may be mentioned that protecting groups such as a methyl group, an ethyl group, an n-propyl group and an n-butyl group are introduced beforehand, with a methyl group and an ethyl group being especially preferable from a standpoint of economic efficiency.

Because there are cases where the process proceeds to (B) without isolating compound (3′) in process (A), the reagents used in the process (B) shall be based on compound (1′). The yield of compound (3′) will not be calculated and only the yield of compound (5′) shall be calculated based on compound (1′).

The amount of compound (4′) to be used is, relative to 1 mole of compound (1′), usually 1 mole to 2 moles, preferably 1.05 moles to 1.75 moles, more preferably 1.05 moles to 1.5 moles.

The reaction solvents used include solvents such as DME, ethylene glycol diethyl ether, ethylene glycol butyl ether, THF and the like, with DME and toluene being especially preferable from a standpoint of reactivity and economic efficiency.

The amount of the solvent to be used is, relative to 1 g of compound (1′), usually 0.5 ml to 500 ml, preferably 0.7 ml to 400 ml, more preferably 1 ml to 350 ml.

The reaction temperature is usually 50° C. to 130° C., preferably 60° C. to 120° C., more preferably 65° C. to 110° C.

The metal catalysts used include Ni, Pd, Pt and the like of Group 10, with Pd being preferable from a standpoint of reactivity. The Pd metal catalysts include, for example, Pd(PPh₃)₄ (PPh₃ represents triphenylphosphine) and Pd(PPh₃)₂Cl₂, with Pd(PPh₃)₂Cl₂ being preferable from a standpoint of economic efficiency and easiness in handling.

The amount of the metal catalyst to be used is, relative to 1 mmole of compound (1′), 20 micromoles to 150 micromoles, preferably 23 micromoles to 145 micromoles, more preferably 25 micromoles to 130 micromoles.

In process (B), the method of charging is not particularly limited but, from a standpoint of safety and operability, it is preferable to charge the solvent, compound (3′) and compound (4′) and, thereafter, to add a metal catalyst, followed by heating.

The reaction time varies depending on the reagent used and the reaction temperature but is usually 0.5 hour to 10 hours, preferably 1 hour to 8 hours, more preferably 1.5 hours to 7 hours.

The degree of progress of the reaction can be confirmed by LC (liquid chromatography).

After allowing the reaction mixture to cool to room temperature, the product may be isolated and purified by a usual aftertreatment.

The purification methods include, for example, column chromatography and a crystallization method. Column chromatography can be carried out by a conventional method to purify the product.

As a method of crystallization, for example, when DME was used as the solvent, the reaction mixture is cooled to room temperature and, thereafter, allowed to stand still or stirred. The temperature at which the mixture is allowed to stand still or stirred is usually −5° C. to 20° C., preferably −2° C. to 15° C., more preferably 0° C. to 10° C.

The time the mixture is allowed to stand still or is stirred varies depending on the solvent used and temperature, but it is desirable to allow the mixture to stand still usually for 1 hour to 48 hours, preferably for 2 hours to 36 hours, more preferably for 3 hours to 25 hours.

Thereafter, by carrying out filtration, the desired compound (5′) in process (B) can be purified.

[Process (C)]

Process (C) is a process wherein the protecting group introduced into the acidic group of compound (5′) is removed to obtain compound (II).

The base used may be either an organic base or an inorganic base. The organic bases include alkylamines, specifically trimethylamine, triethylamine, tripropylamine and the like, with triethylamine being the most preferable from a standpoint of economic efficiency. Inorganic bases include hydroxides of alkali metals and alkaline earth metals; carbonates and hydrogen carbonates of alkali metals; alkoxides of alkali metals; and the like. Specifically, there may be mentioned bases such as sodium hydroxide, potassium hydroxide, sodium hydrogen carbonate, potassium hydrogen carbonate, lithium hydroxide, and sodium methoxide. Of these, lithium hydroxide, sodium hydrogen carbonate, and sodium methoxide are preferable from a standpoint of reactivity, with lithium hydroxide being especially preferable from a standpoint of handling property.

The amount of the base to be used is, relative to 1 mole of compound (5′), usually 1 mole to 7 moles, preferably 1.1 moles to 5.5 moles, more preferably 1.1 moles to 5 moles.

The reaction solvents used include solvents such as methanol, ethanol, i-propyl alcohol, t-butyl alcohol, n-butanol, THF, and N,N-dimethylformamide (hereinafter, abbreviated as DMF). Among these, methanol, ethanol, and i-propyl alcohol are preferable from a standpoint of reactivity, and methanol and ethanol are especially preferable from a standpoint of economic efficiency.

The solvent is used, relative to 1 g of compound (5′), usually in an amount of 0.5 ml to 1,500 ml, preferably 0.7 ml to 1,400 ml, more preferably 1 ml to 1,300 ml.

The reaction temperature is usually 50° C. to 100° C., preferably 60° C. to 95° C., more preferably 65° C. to 90° C.

In the process (C), the order of charging is not particularly limited but, from a standpoint of safety and operability, it is preferable to charge the solvent, compound (5′) and base, followed by heating.

The reaction time varies depending on the reagents used and the reaction temperature but is usually 0.5 hour to 15 hours, preferably 1 hour to 14 hours, more preferably 1.5 hours to 13 hours.

The degree of progress of the reaction can be confirmed by LC (liquid chromatography).

The reaction mixture may be used in the next process without separation and purification after distilling off the solvent under reduced pressure. Alternatively, after cooling the reaction mixture to room temperature, the product may be isolated and purified by a usual aftertreatment. For example, column chromatography may be mentioned. By carrying out column chromatography by a usual method, the product can be purified.

When purification of compound (II′) is difficult because of troublesome handling, it is possible that process (C) is not carried out but the compound is coordinated to a metal and thereafter the protecting group is removed.

Specific examples of compound (II′) include compounds (II′-1) to (II′-75), represented by the following formula and Table 3-1 to Table 3-4.

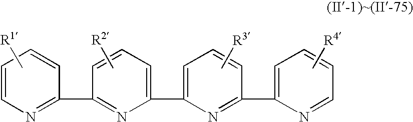

TABLE 3-1

| Com- | R¹ | | | | | | | R² | | | | | | |
|---|---|---|---|---|---|---|---|---|---|---|---|---|---|---|
| pound | Position | a′ | b′ | p′ | L′ | Ar | R^{5′} | Position | a′ | b′ | p′ | L′ | Ar | R^{5′} |
| II′-1 | 4 | 0 | 0 | — | — | — | —COOH | 4′ | 0 | 0 | — | — | — | —COOH |
| II′-2 | 4 | 0 | 0 | — | — | — | —COOH | 4′ | 0 | 0 | — | — | — | —COOH |
| II′-3 | 4 | 0 | 0 | — | — | — | —COOH | 4′ | 0 | 0 | — | — | — | —COOH |
| II′-4 | 4 | 0 | 0 | — | — | — | —COOH | 4′ | 0 | 0 | — | — | — | —COOH |
| II′-5 | 4 | 0 | 0 | — | — | — | —COOH | 4′ | 0 | 0 | — | — | — | —COOH |
| II′-6 | 4 | 0 | 0 | — | — | — | —COOH | 4′ | 0 | 0 | — | — | — | —COOH |
| II′-7 | 4 | 0 | 0 | — | — | — | —COOH | 4′ | 0 | 0 | — | — | — | —COOH |
| II′-8 | 4 | 0 | 0 | — | — | — | —COOH | 4′ | 0 | 0 | — | — | — | —COOH |
| II′-9 | 4 | 0 | 0 | — | — | — | —COOH | 4′ | 0 | 0 | — | — | — | —COOH |
| II′-10 | 4 | 0 | 0 | — | — | — | —COOH | 4′ | 0 | 0 | — | — | — | —COOH |
| II′-11 | 4 | 0 | 0 | — | — | — | —COOH | 4′ | 0 | 0 | — | — | — | —COOH |
| II′-12 | 4 | 0 | 0 | — | — | — | —COOH | 4′ | 0 | 0 | — | — | — | —COOH |
| II′-13 | 4 | 0 | 0 | — | — | — | —COOH | 4′ | 0 | 0 | — | — | — | —COOH |
| II′-14 | 4 | 0 | 0 | — | — | — | —COOH | 4′ | 0 | 0 | — | — | — | —COOH |
| II′-15 | 4 | 0 | 0 | — | — | — | —COOH | 4′ | 0 | 0 | — | — | — | —COOH |
| II′-16 | 4 | 0 | 0 | — | — | — | —COOH | 4′ | 0 | 0 | — | — | — | —COOH |
| II′-17 | 4 | 0 | 0 | — | — | — | —COOH | 4′ | 0 | 0 | — | — | — | —COOH |
| II′-18 | 4 | 0 | 0 | — | — | — | —COOH | 4′ | 0 | 0 | — | — | — | —COOH |
| II′-19 | 4 | 0 | 0 | — | — | — | —COOH | 4′ | 0 | 0 | — | — | — | —COOH |
| II′-20 | 4 | 0 | 0 | — | — | — | —COOH | 4′ | 0 | 0 | — | — | — | —COOH |
| II′-21 | 4 | 0 | 0 | — | — | — | —COOH | 4′ | 0 | 0 | — | — | — | —COOH |
| II′-22 | 4 | 0 | 0 | — | — | — | —COOH | 4′ | 0 | 0 | — | — | — | —COOH |
| II′-23 | 4 | 0 | 0 | — | — | — | —COOH | 4′ | 0 | 0 | — | — | — | —COOH |
| II′-24 | 4 | 0 | 0 | — | — | — | —COOH | 4′ | 0 | 0 | — | — | — | —COOH |
| II′-25 | 4 | 1 | 1 | 1 | —(CH═CH)— | A-1 | —OMe | 4′ | 0 | 0 | — | — | — | —COOH |
| II′-26 | 4 | 1 | 1 | 1 | —(CH═CH)— | A-1 | —OC10H21 | 4′ | 0 | 0 | — | — | — | —COOH |
| II′-27 | 4 | 1 | 1 | 1 | —(CH═CH)— | A-2 | —OMe | 4′ | 0 | 0 | — | — | — | —COOH |
| II′-28 | 4 | 1 | 1 | 1 | —(CH═CH)— | A-4 | —OMe | 4′ | 0 | 0 | — | — | — | —COOH |
| II′-29 | 4 | 1 | 1 | 1 | —(CH═CH)— | A-4 | —C10H21 | 4′ | 0 | 0 | — | — | — | —COOH |
| II′-30 | 4 | 1 | 1 | 1 | —(CH═CH)— | A-5 | —OMe | 4′ | 0 | 0 | — | — | — | —COOH |
| II′-31 | 4 | 0 | 0 | — | — | — | —COOH | 4′ | 0 | 0 | — | — | — | —COOH |
| II′-32 | 4 | 0 | 0 | — | — | — | —COOH | 4′ | 0 | 0 | — | — | — | —COOH |
| II′-33 | 4 | 0 | 0 | — | — | — | —COOH | 4′ | 0 | 0 | — | — | — | —COOH |
| II′-34 | 4 | 0 | 0 | — | — | — | —COOH | 4′ | 0 | 0 | — | — | — | —COOH |
| II′-35 | 4 | 0 | 0 | — | — | — | —COOH | 4′ | 0 | 0 | — | — | — | —COOH |
| II′-36 | 4 | 0 | 0 | — | — | — | —COOH | 4′ | 0 | 0 | — | — | — | —COOH |
| II′-37 | 4 | 0 | 0 | — | — | — | —COOH | 4′ | 1 | 1 | 1 | —(CH═CH)— | A-1 | —OMe |
| II′-38 | 4 | 0 | 0 | — | — | — | —COOH | 4′ | 1 | 1 | 1 | —(CH═CH)— | A-1 | —OC10H21 |

TABLE 3-2

| Com- | R³ | | | | | | | R⁴ | | | | | | |
|---|---|---|---|---|---|---|---|---|---|---|---|---|---|---|
| pound | Position | a′ | b′ | p′ | L′ | Ar | R^{5′} | Position | a′ | b′ | p′ | L′ | Ar | R^{5′} |
| II′-1 | 4″ | 1 | 1 | 1 | —(CH═CH)— | A-1 | —OMe | 4′″ | 0 | 0 | — | — | — | —COOH |
| II′-2 | 4″ | 1 | 1 | 1 | —(CH═CH)— | A-1 | —OC10H21 | 4′″ | 0 | 0 | — | — | — | —COOH |
| II′-3 | 4″ | 1 | 1 | 1 | —(CH═CH)— | A-2 | —OMe | 4′″ | 0 | 0 | — | — | — | —COOH |
| II′-4 | 4″ | 1 | 1 | 1 | —(CH═CH)— | A-3 | —OMe | 4′″ | 0 | 0 | — | — | — | —COOH |
| II′-5 | 4″ | 1 | 1 | 1 | —(CH═CH)— | A-4 | —OMe | 4′″ | 0 | 0 | — | — | — | —COOH |
| II′-6 | 4″ | 1 | 1 | 1 | —(CH═CH)— | A-4 | —C10H21 | 4′″ | 0 | 0 | — | — | — | —COOH |
| II′-7 | 4″ | 1 | 1 | 1 | —(CH═CH)— | A-5 | —OMe | 4′″ | 0 | 0 | — | — | — | —COOH |
| II′-8 | 4″ | 1 | 1 | 1 | —(CH═CH)— | A-6 | —OMe | 4′″ | 0 | 0 | — | — | — | —COOH |
| II′-9 | 4″ | 1 | 1 | 1 | —(CH═CH)— | A-7 | —OMe | 4′″ | 0 | 0 | — | — | — | —COOH |
| II′-10 | 4″ | 1 | 1 | 1 | —(CH═CH)— | A-8 | —OMe | 4′″ | 0 | 0 | — | — | — | —COOH |
| II′-11 | 4″ | 1 | 1 | 1 | —(CH═CH)— | A-9 | —OMe | 4′″ | 0 | 0 | — | — | — | —COOH |
| II′-12 | 4″ | 1 | 1 | 1 | —(CH═CH)— | A-10 | —OMe | 4′″ | 0 | 0 | — | — | — | —COOH |
| II′-13 | 4″ | 1 | 1 | 1 | —(CH═CH)— | A-11 | —OMe | 4′″ | 0 | 0 | — | — | — | —COOH |
| II′-14 | 4″ | 1 | 1 | 1 | —(CH═CH)— | A-12 | —OMe | 4′″ | 0 | 0 | — | — | — | —COOH |
| II′-15 | 4″ | 1 | 1 | 1 | —(CH═CH)— | A-13 | —OMe | 4′″ | 0 | 0 | — | — | — | —COOH |
| II′-16 | 4″ | 1 | 1 | 1 | —(CH═CH)— | A-14 | —OMe | 4′″ | 0 | 0 | — | — | — | —COOH |
| II′-17 | 4″ | 1 | 1 | 1 | —(CH═CH)— | A-15 | —OMe | 4′″ | 0 | 0 | — | — | — | —COOH |
| II′-18 | 4″ | 1 | 1 | 1 | —(CH═CH)— | A-16 | —OMe | 4′″ | 0 | 0 | — | — | — | —COOH |
| II′-19 | 4″ | 1 | 1 | 1 | —(CH═CH)— | A-17 | —OMe | 4′″ | 0 | 0 | — | — | — | —COOH |
| II′-20 | 4″ | 1 | 1 | 1 | —(CH═CH)— | A-18 | —OMe | 4′″ | 0 | 0 | — | — | — | —COOH |
| II′-21 | 4″ | 1 | 1 | 1 | —(CH═CH)— | A-19 | —OMe | 4′″ | 0 | 0 | — | — | — | —COOH |
| II′-22 | 4″ | 1 | 1 | 1 | —(CH═CH)— | A-20 | —OMe | 4′″ | 0 | 0 | — | — | — | —COOH |
| II′-23 | 4″ | 1 | 1 | 1 | —(CH═CH)— | A-21 | —OMe | 4′″ | 0 | 0 | — | — | — | —COOH |
| II′-24 | 4″ | 1 | 1 | 1 | —(CH═CH)— | A-22 | OMe | 4′″ | 0 | 0 | — | — | — | —COOH |
| II′-25 | 4″ | 0 | 0 | — | — | — | —COOH | 4′″ | 1 | 1 | 1 | —(CH═CH)— | A-1 | —OMe |
| II′-26 | 4″ | 0 | 0 | — | — | — | —COOH | 4′″ | 1 | 1 | 1 | —(CH═CH)— | A-1 | —OC10H21 |
| II′-27 | 4″ | 0 | 0 | — | — | — | —COOH | 4′″ | 1 | 1 | 1 | —(CH═CH)— | A-2 | —OMe |
| II′-28 | 4″ | 0 | 0 | — | — | — | —COOH | 4′″ | 1 | 1 | 1 | —(CH═CH)— | A-4 | —OMe |
| II′-29 | 4″ | 0 | 0 | — | — | — | —COOH | 4′″ | 1 | 1 | 1 | —(CH═CH)— | A-4 | —C10H21 |
| II′-30 | 4″ | 0 | 0 | — | — | — | —COOH | 4′″ | 1 | 1 | 1 | —(CH═CH)— | A-5 | —OMe |
| II′-31 | 4″ | 0 | 0 | — | — | — | H | 4′″ | 1 | 1 | 1 | —(CH═CH)— | A-1 | —OMe |
| II′-32 | 4″ | 0 | 0 | — | — | — | H | 4′″ | 1 | 1 | 1 | —(CH═CH)— | A-1 | —OC10H21 |
| II′-33 | 4″ | 0 | 0 | — | — | — | H | 4′″ | 1 | 1 | 1 | —(CH═CH)— | A-2 | —OMe |
| II′-34 | 4″ | 0 | 0 | — | — | — | H | 4′″ | 1 | 1 | 1 | —(CH═CH)— | A-4 | —OMe |
| II′-35 | 4″ | 0 | 0 | — | — | — | H | 4′″ | 1 | 1 | 1 | —(CH═CH)— | A-4 | —C10H21 |
| II′-36 | 4″ | 0 | 0 | — | — | — | H | 4′″ | 1 | 1 | 1 | —(CH═CH)— | A-5 | —OMe |
| II′-37 | 4″ | 1 | 1 | 1 | —(CH═CH)— | A-1 | —OMe | 4′″ | 0 | 0 | — | — | — | —COOH |
| II′-38 | 4″ | 1 | 1 | 1 | —(CH═CH)— | A-1 | —OC10H21 | 4′″ | 0 | 0 | — | — | — | —COOH |

TABLE 3-3

| Com- | R¹ | | | | | | | R² | | | | | | |
|---|---|---|---|---|---|---|---|---|---|---|---|---|---|---|
| pound | Position | a′ | b′ | p′ | L′ | Ar | R^{5′} | Position | a′ | b′ | p′ | L′ | Ar | R^{5′} |
| II′-39 | 4 | 0 | 0 | — | — | — | —COOH | 4′ | 1 | 1 | 1 | —(CH═CH)— | A-2 | —OMe |
| II′-40 | 4 | 0 | 0 | — | — | — | —COOH | 4′ | 1 | 1 | 1 | —(CH═CH)— | A-4 | —OMe |
| II′-41 | 4 | 0 | 0 | — | — | — | —COOH | 4′ | 1 | 1 | 1 | —(CH═CH)— | A-4 | —C10H21 |
| II′-42 | 4 | 0 | 0 | — | — | — | —COOH | 4′ | 1 | 1 | 1 | —(CH═CH)— | A-5 | —OMe |
| II′-43 | 4 | 0 | 0 | — | — | — | —COOH | 4′ | 0 | 0 | — | — | — | —COOH |
| II′-44 | 4 | 0 | 0 | — | — | — | —COOH | 4′ | 0 | 0 | — | — | — | —COOH |
| II′-45 | 4 | 0 | 0 | — | — | — | —COOH | 4′ | 0 | 0 | — | — | — | —COOH |
| II′-46 | 4 | 0 | 0 | — | — | — | —COOH | 4′ | 0 | 0 | — | — | — | —COOH |
| II′-47 | 4 | 0 | 0 | — | — | — | —COOH | 4′ | 0 | 0 | — | — | — | —COOH |
| II′-48 | 4 | 0 | 0 | — | — | — | —COOH | 4′ | 0 | 0 | — | — | — | —COOH |
| II′-49 | 4 | 0 | 0 | — | — | — | —COOH | 4′ | 0 | 0 | — | — | — | —COOH |
| II′-50 | 4 | 0 | 0 | — | — | — | —COOH | 4′ | 0 | 0 | — | — | — | —COOH |
| II′-51 | 4 | 0 | 0 | — | — | — | —COOH | 4′ | 0 | 0 | — | — | — | —COOH |
| II′-52 | 4 | 0 | 0 | — | — | — | —COOH | 4′ | 0 | 0 | — | — | — | —COOH |
| II′-53 | 4 | 0 | 0 | — | — | — | —COOH | 4′ | 0 | 0 | — | — | — | —COOH |
| II′-54 | 4 | 0 | 0 | — | — | — | —COOH | 4′ | 0 | 0 | — | — | — | —COOH |
| II′-55 | 4 | 0 | 0 | — | — | — | —COOH | 4′ | 0 | 0 | — | — | — | —COOH |
| II′-56 | 4 | 0 | 0 | — | — | — | —COOH | 4′ | 0 | 0 | — | — | — | —COOH |
| II′-57 | 4 | 0 | 0 | — | — | — | —COOH | 4′ | 0 | 0 | — | — | — | —COOH |
| II′-58 | 4 | 0 | 0 | — | — | — | —COOH | 4′ | 0 | 0 | — | — | — | —COOH |
| II′-59 | 4 | 1 | 0 | 1 | —(CH═CH)— | — | —COOH | 4′ | 0 | 0 | — | — | — | —Me |
| II′-60 | 4 | 1 | 0 | 1 | —(CH═CH)— | — | —COOH | 4′ | 0 | 0 | — | — | — | —C10H21 |
| II′-61 | 4 | 1 | 0 | 1 | —(CH═CH)— | — | —COOH | 4′ | 0 | 0 | — | — | — | —H |
| II′-62 | 4 | 1 | 0 | 1 | —(CH═CH)— | — | —COOH | 4′ | 0 | 0 | — | — | — | —OMe |
| II′-63 | 4 | 1 | 0 | 1 | —(CH═CH)— | — | —COOH | 4′ | 0 | 0 | — | — | — | —N(Me)2 |
| II′-64 | 4 | 1 | 0 | 1 | —(CH═CH)— | — | —COOH | 4′ | 0 | 0 | — | — | — | —COOH |
| II′-65 | 4 | 0 | 0 | — | — | — | —SO3H | 4′ | 0 | 0 | — | — | — | —SO3H |
| II′-66 | 4 | 0 | 0 | — | — | — | —PO3H2 | 4′ | 0 | 0 | — | — | — | —PO3H2 |
| II′-67 | 4 | 0 | 0 | — | — | — | —COOH | 4′ | 0 | 0 | — | — | — | —COOH |
| II′-68 | 4 | 0 | 0 | — | — | — | —COOH | 4′ | 1 | 1 | 1 | —(CH═CH)— | A-1 | —COOH |
| II′-69 | 4 | 1 | 1 | 1 | —(CH═CH)— | A-1 | —COOH | 4′ | 1 | 1 | 1 | —(CH═CH)— | A-1 | —COOH |
| II′-70 | 4 | 1 | 1 | 1 | —(CH═CH)— | A-1 | —COOH | 4′ | 1 | 1 | 1 | —(CH═CH)— | A-1 | —COOH |
| II′-71 | 4 | 1 | 1 | 1 | —(CH═CH)— | A-4 | —COOH | 4′ | 1 | 1 | 1 | —(CH═CH)— | A-4 | —COOH |
| II′-72 | 4 | 1 | 1 | 1 | —(CH═CH)— | A-4 | —COOH | 4′ | 1 | 1 | 1 | —(CH═CH)— | A-4 | —COOH |
| II′-73 | 4 | 1 | 1 | 1 | —(CH═CH)— | A-1 | —COOH | 4′ | 1 | 1 | 1 | —(CH═CH)— | A-1 | —COOH |
| II′-74 | 4 | 1 | 1 | 1 | —(CH═CH)— | A-4 | —COOH | 4′ | 1 | 1 | 1 | —(CH═CH)— | A-4 | —COOH |

TABLE 3-4

| Com- | R³ | | | | | | | R⁴ | | | | | | |
|---|---|---|---|---|---|---|---|---|---|---|---|---|---|---|
| pound | Position | a′ | b′ | p′ | L′ | Ar | R^{5′} | Position | a′ | b′ | p′ | L′ | Ar | R^{5′} |
| II′-39 | 4″ | 1 | 1 | 1 | —(CH═CH)— | A-2 | —OMe | 4′″ | 0 | 0 | — | — | — | —COOH |
| II′-40 | 4″ | 1 | 1 | 1 | —(CH═CH)— | A-4 | —OMe | 4′″ | 0 | 0 | — | — | — | —COOH |
| II′-41 | 4″ | 1 | 1 | 1 | —(CH═CH)— | A-4 | —C10H21 | 4′″ | 0 | 0 | — | — | — | —COOH |
| II′-42 | 4″ | 1 | 1 | 1 | —(CH═CH)— | A-5 | —OMe | 4′″ | 0 | 0 | — | — | — | —COOH |
| II′-43 | 4″ | 0 | 0 | — | — | — | —OMe | 4′″ | 1 | 1 | 1 | —(CH═CH)— | A-1 | —OMe |
| II′-44 | 4″ | 0 | 0 | — | — | — | —OC10H21 | 4′″ | 1 | 1 | 1 | —(CH═CH)— | A-1 | —OC10H21 |
| II′-45 | 4″ | 0 | 0 | — | — | — | —OMe | 4′″ | 1 | 1 | 1 | —(CH═CH)— | A-1 | —OC10H21 |
| II′-46 | 4″ | 0 | 0 | — | — | — | —OC10H21 | 4′″ | 1 | 1 | 1 | —(CH═CH)— | A-1 | —OMe |
| II′-47 | 4″ | 0 | 0 | — | — | — | —OMe | 4′″ | 1 | 1 | 1 | —(CH═CH)— | A-2 | —OMe |
| II′-48 | 4″ | 0 | 0 | — | — | — | —OMe | 4′″ | 1 | 1 | 1 | —(CH═CH)— | A-4 | —OMe |
| II′-49 | 4″ | 0 | 0 | — | — | — | —OC10H21 | 4′″ | 1 | 1 | 1 | —(CH═CH)— | A-4 | —C10H21 |
| II′-50 | 4″ | 0 | 0 | — | — | — | —OMe | 4′″ | 1 | 1 | 1 | —(CH═CH)— | A-4 | —C10H21 |
| II′-51 | 4″ | 0 | 0 | — | — | — | —OC10H21 | 4′″ | 1 | 1 | 1 | —(CH═CH)— | A-4 | —OMe |
| II′-52 | 4″ | 0 | 0 | — | — | — | —OMe | 4′″ | 1 | 1 | 1 | —(CH═CH)— | A-5 | —OMe |
| II′-53 | 4″ | 0 | 0 | — | — | — | —Me | 4′″ | 1 | 1 | 1 | —(CH═CH)— | A-4 | —COOH |
| II′-54 | 4″ | 0 | 0 | — | — | — | —C10H21 | 4′″ | 1 | 1 | 1 | —(CH═CH)— | A-4 | —COOH |
| II′-55 | 4″ | 0 | 0 | — | — | — | —H | 4′″ | 1 | 1 | 1 | —(CH═CH)— | A-4 | —COOH |
| II′-56 | 4″ | 0 | 0 | — | — | — | —OMe | 4′″ | 1 | 1 | 1 | —(CH═CH)— | A-4 | —COOH |
| II′-57 | 4″ | 0 | 0 | — | — | — | —N(Me)2 | 4′″ | 1 | 1 | 1 | —(CH═CH)— | A-4 | —COOH |
| II′-58 | 4″ | 0 | 0 | — | — | — | —COOH | 4′″ | 1 | 1 | 1 | —(CH═CH)— | A-4 | —COOH |
| II′-59 | 4″ | 0 | 0 | — | — | — | —Me | 4′″ | 1 | 0 | 1 | —(CH═CH)— | — | —COOH |
| II′-60 | 4″ | 0 | 0 | — | — | — | —C10H21 | 4′″ | 1 | 0 | 1 | —(CH═CH)— | — | —COOH |
| II′-61 | 4″ | 0 | 0 | — | — | — | —H | 4′″ | 1 | 0 | 1 | —(CH═CH)— | — | —COOH |
| II′-62 | 4″ | 0 | 0 | — | — | — | —OMe | 4′″ | 1 | 0 | 1 | —(CH═CH)— | — | —COOH |
| II′-63 | 4″ | 0 | 0 | — | — | — | —N(Me)2 | 4′″ | 1 | 0 | 1 | —(CH═CH)— | — | —COOH |
| II′-64 | 4″ | 0 | 0 | — | — | — | —COOH | 4′″ | 1 | 0 | 1 | —(CH═CH)— | — | —COOH |
| II′-65 | 4″ | 1 | 1 | 1 | —(CH═CH)— | A-1 | —OMe | 4′″ | 0 | 0 | — | — | — | —SO3H |
| II′-66 | 4″ | 1 | 1 | 1 | —(CH═CH)— | A-1 | —OMe | 4′″ | 0 | 0 | — | — | — | —PO3H2 |
| II′-67 | 4″ | 1 | 1 | 1 | —(C≡C)— | A-1 | —OMe | 4′″ | 0 | 0 | — | — | — | —COOH |
| II′-68 | 4″ | 0 | 0 | — | — | — | —COOH | 4′″ | 1 | 1 | 1 | —(CH═CH)— | A-1 | —H |
| II′-69 | 4″ | 1 | 0 | 1 | —(CH═CH)— | — | —Me | 4′″ | 1 | 0 | 1 | —(CH═CH)— | — | —Me |
| II′-70 | 4″ | 1 | 0 | 1 | —(C≡C)— | — | —H | 4′″ | 1 | 0 | 1 | —(C≡C)— | — | —H |
| II′-71 | 4″ | 1 | 0 | 1 | —(CH═CH)— | — | —Me | 4′″ | 1 | 0 | 1 | —(CH═CH)— | — | —Me |
| II′-72 | 4″ | 1 | 0 | 1 | —(C≡C)— | — | —H | 4′″ | 1 | 0 | 1 | —(C≡C)— | — | —H |
| II′-73 | 4″ | 1 | 1 | 1 | —(CH═CH)— | A-1 | —OMe | 4′″ | 1 | 1 | 1 | —(CH═CH)— | A-1 | —COOH |
| II′-74 | 4″ | 1 | 1 | 1 | —(CH═CH)— | A-1 | —OMe | 4′″ | 1 | 1 | 1 | —(CH═CH)— | A-4 | —COOH |
| II′-75 | 4″ | 1 | 1 | 1 | —(CH═CH)— | A-1 | —OMe | 4′″ | 1 | 1 | 1 | —(CH═CH)— | A-1 | —OMe |

Complex compound (I′) of the present invention is obtained by having the compound (II′) coordinated to a metal atom.

In addition, complex compound (I′) of the present invention comprises a metal atom as the central atom and a compound represented by the formula (II′) as one of the ligands.

There may be other ligands coordinated than the compound represented by the formula (II′). Other ligands contained in complex compound (I′) include, for example, isothiocyanate (13 N═C═S, hereinafter may sometimes be referred to as NCS), thiocyanate (—S—C≡N, hereinafter may sometimes be referred to as SCN), diketonate, chloro, bromo, iodo, cyano and a hydroxyl group, with NCS or SCN being preferable. The complex compound may exist accompanied with counter anions such as halogen anions, in a form with the charge neutralized.

The method for manufacturing complex compound (I′) is the same as in the complex compound (I) when the metal atom is Ru.

Specific examples of complex compound (I′) include the compounds (I′-1) to (I′-258) represented by the following formula and Table 4-1 to Table 4-7:

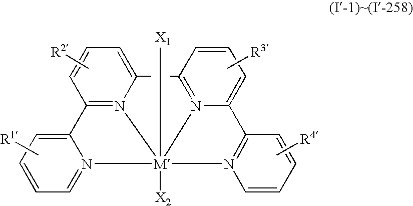

TABLE 4-1

| Compound | M′ | Compound (II′) | X₁ = X₂ |
|---|---|---|---|
| I′-1 | Ru | II′-1 | —NCS |
| I′-2 | Ru | II′-2 | —NCS |
| I′-3 | Ru | II′-3 | —NCS |
| I′-4 | Ru | II′-4 | —NCS |
| I′-5 | Ru | II′-5 | —NCS |
| I′-6 | Ru | II′-6 | —NCS |
| I′-7 | Ru | II′-7 | —NCS |
| I′-8 | Ru | II′-8 | —NCS |
| I′-9 | Ru | II′-9 | —NCS |
| I′-10 | Ru | II′-10 | —NCS |
| I′-11 | Ru | II′-11 | —NCS |
| I′-12 | Ru | II′-12 | —NCS |
| I′-13 | Ru | II′-13 | —NCS |
| I′-14 | Ru | II′-14 | —NCS |
| I′-15 | Ru | II′-15 | —NCS |
| I′-16 | Ru | II′-16 | —NCS |
| I′-17 | Ru | II′-17 | —NCS |
| I′-18 | Ru | II′-18 | —NCS |
| I′-19 | Ru | II′-19 | —NCS |
| I′-20 | Ru | II′-20 | —NCS |
| I′-21 | Ru | II′-21 | —NCS |
| I′-22 | Ru | II′-22 | —NCS |
| I′-23 | Ru | II′-23 | —NCS |
| I′-24 | Ru | II′-24 | —NCS |
| I′-25 | Ru | II′-25 | —NCS |
| I′-26 | Ru | II′-26 | —NCS |
| I′-27 | Ru | II′-27 | —NCS |
| I′-28 | Ru | II′-28 | —NCS |
| I′-29 | Ru | II′-29 | —NCS |
| I′-30 | Ru | II′-30 | —NCS |
| I′-31 | Ru | II′-31 | —NCS |
| I′-32 | Ru | II′-32 | —NCS |
| I′-33 | Ru | II′-33 | —NCS |
| I′-34 | Ru | II′-34 | —NCS |
| I′-35 | Ru | II′-35 | —NCS |
| I′-36 | Ru | II′-36 | —NCS |
| I′-37 | Ru | II′-37 | —NCS |
| I′-38 | Ru | II′-38 | —NCS |
| I′-39 | Ru | II′-39 | —NCS |
| I′-40 | Ru | II′-40 | —NCS |

TABLE 4-2

| Compound | M′ | Compound (II′) | X₁ = X₂ |
|---|---|---|---|
| I′-41 | Ru | II′-41 | —NCS |
| I′-42 | Ru | II′-42 | —NCS |
| I′-43 | Ru | II′-43 | —NCS |
| I′-44 | Ru | II′-44 | —NCS |
| I′-45 | Ru | II′-45 | —NCS |
| I′-46 | Ru | II′-46 | —NCS |
| I′-47 | Ru | II′-47 | —NCS |
| I′-48 | Ru | II′-48 | —NCS |
| I′-49 | Ru | II′-49 | —NCS |
| I′-50 | Ru | II′-50 | —NCS |
| I′-51 | Ru | II′-51 | —NCS |
| I′-52 | Ru | II′-52 | —NCS |
| I′-53 | Ru | II′-53 | —NCS |
| I′-54 | Ru | II′-54 | —NCS |
| I′-55 | Ru | II′-55 | —NCS |
| I′-56 | Ru | II′-56 | —NCS |
| I′-57 | Ru | II′-57 | —NCS |
| I′-58 | Ru | II′-58 | —NCS |
| I′-59 | Ru | II′-59 | —NCS |
| I′-60 | Ru | II′-60 | —NCS |
| I′-61 | Ru | II′-61 | —NCS |
| I′-62 | Ru | II′-62 | —NCS |
| I′-63 | Ru | II′-63 | —NCS |
| I′-64 | Ru | II′-64 | —NCS |
| I′-65 | Ru | II′-65 | —NCS |
| I′-66 | Ru | II′-66 | —NCS |
| I′-67 | Ru | II′-67 | —NCS |
| I′-68 | Ru | II′-68 | —NCS |
| I′-69 | Ru | II′-69 | —NCS |
| I′-70 | Ru | II′-70 | —NCS |
| I′-71 | Ru | II′-71 | —NCS |
| I′-72 | Ru | II′-72 | —NCS |
| I′-73 | Ru | II′-73 | —NCS |
| I′-74 | Ru | II′-74 | —NCS |
| I′-75 | Ru | II′-75 | —NCS |
| I′-76 | Ru | II′-1 | —SCN |
| I′-77 | Ru | II′-2 | —SCN |
| I′-78 | Ru | II′-3 | —SCN |
| I′-79 | Ru | II′-4 | —SCN |
| I′-80 | Ru | II′-5 | —SCN |

TABLE 4-3

| Compound | M′ | Compound (II′) | X₁ = X₂ |
|---|---|---|---|
| I′-81 | Ru | II′-6 | —SCN |
| I′-82 | Ru | II′-7 | —SCN |
| I′-83 | Ru | II′-8 | —SCN |
| I′-84 | Ru | II′-9 | —SCN |
| I′-85 | Ru | II′-10 | —SCN |
| I′-86 | Ru | II′-11 | —SCN |
| I′-87 | Ru | II′-12 | —SCN |
| I′-88 | Ru | II′-13 | —SCN |
| I′-89 | Ru | II′-14 | —SCN |
| I′-90 | Ru | II′-15 | —SCN |
| I′-91 | Ru | II′-16 | —SCN |
| I′-92 | Ru | II′-17 | —SCN |
| I′-93 | Ru | II′-18 | —SCN |
| I′-94 | Ru | II′-19 | —SCN |
| I′-95 | Ru | II′-20 | —SCN |
| I′-96 | Ru | II′-21 | —SCN |
| I′-97 | Ru | II′-22 | —SCN |
| I′-98 | Ru | II′-23 | —SCN |
| I′-99 | Ru | II′-24 | —SCN |
| I′-100 | Ru | II′-25 | —SCN |
| I′-101 | Ru | II′-26 | —SCN |
| I′-102 | Ru | II′-27 | —SCN |
| I′-103 | Ru | II′-28 | —SCN |
| I′-104 | Ru | II′-29 | —SCN |
| I′-105 | Ru | II′-30 | —SCN |
| I′-106 | Ru | II′-31 | —SCN |
| I′-107 | Ru | II′-32 | —SCN |
| I′-108 | Ru | II′-33 | —SCN |
| I′-109 | Ru | II′-34 | —SCN |
| I′-110 | Ru | II′-35 | —SCN |
| I′-111 | Ru | II′-36 | —SCN |
| I′-112 | Ru | II′-37 | —SCN |
| I′-113 | Ru | II′-38 | —SCN |
| I′-114 | Ru | II′-39 | —SCN |
| I′-115 | Ru | II′-40 | —SCN |
| I′-116 | Ru | II′-41 | —SCN |
| I′-117 | Ru | II′-42 | —SCN |
| I′-118 | Ru | II′-43 | —SCN |
| I′-119 | Ru | II′-44 | —SCN |
| I′-120 | Ru | II′-45 | —SCN |

TABLE 4-4

| Compound | M′ | Compound (II′) | X₁ = X₂ |
|---|---|---|---|
| I′-121 | Ru | II′-46 | —SCN |
| I′-122 | Ru | II′-47 | —SCN |
| I′-123 | Ru | II′-48 | —SCN |
| I′-124 | Ru | II′-49 | —SCN |
| I′-125 | Ru | II′-50 | —SCN |
| I′-126 | Ru | II′-51 | —SCN |
| I′-127 | Ru | II′-52 | —SCN |
| I′-128 | Ru | II′-53 | —SCN |
| I′-129 | Ru | II′-54 | —SCN |
| I′-130 | Ru | II′-55 | —SCN |
| I′-131 | Ru | II′-56 | —SCN |
| I′-132 | Ru | II′-57 | —SCN |
| I′-133 | Ru | II′-58 | —SCN |
| I′-134 | Ru | II′-59 | —SCN |
| I′-135 | Ru | II′-60 | —SCN |
| I′-136 | Ru | II′-61 | —SCN |
| I′-137 | Ru | II′-62 | —SCN |
| I′-138 | Ru | II′-63 | —SCN |
| I′-139 | Ru | II′-64 | —SCN |
| I′-140 | Ru | II′-65 | —SCN |
| I′-141 | Ru | II′-66 | —SCN |
| I′-142 | Ru | II′-67 | —SCN |
| I′-143 | Ru | II′-68 | —SCN |
| I′-144 | Ru | II′-69 | —SCN |
| I′-145 | Ru | II′-70 | —SCN |
| I′-146 | Ru | II′-71 | —SCN |
| I′-147 | Ru | II′-72 | —SCN |
| I′-148 | Ru | II′-73 | —SCN |
| I′-149 | Ru | II′-74 | —SCN |
| I′-150 | Ru | II′-75 | —SCN |
| I′-151 | Ru | II′-1 | —CN |
| I′-152 | Ru | II′-2 | —CN |
| I′-153 | Ru | II′-5 | —CN |
| I′-154 | Ru | II′-6 | —CN |
| I′-155 | Ru | II′-25 | —CN |
| I′-156 | Ru | II′-26 | —CN |
| I′-157 | Ru | II′-27 | —CN |
| I′-158 | Ru | II′-28 | —CN |
| I′-159 | Ru | II′-29 | —CN |
| I′-160 | Ru | II′-31 | —CN |

TABLE 4-5

| Compound | M′ | Compound (II′) | X₁ = X₂ |
|---|---|---|---|
| I′-161 | Ru | II′-32 | —CN |
| I′-162 | Ru | II′-34 | —CN |
| I′-163 | Ru | II′-35 | —CN |
| I′-164 | Ru | II′-37 | —CN |
| I′-165 | Ru | II′-43 | —CN |
| I′-166 | Ru | II′-44 | —CN |
| I′-167 | Ru | II′-45 | —CN |
| I′-168 | Ru | II′-46 | —CN |
| I′-169 | Ru | II′-48 | —CN |
| I′-170 | Ru | II′-49 | —CN |
| I′-171 | Ru | II′-53 | —CN |
| I′-172 | Ru | II′-54 | —CN |
| I′-173 | Ru | II′-55 | —CN |
| I′-174 | Ru | II′-59 | —CN |
| I′-175 | Ru | II′-60 | —CN |
| I′-176 | Ru | II′-61 | —CN |
| I′-177 | Ru | II′-75 | —CN |
| I′-178 | Ru | II′-1 | —Cl |
| I′-179 | Ru | II′-2 | —Cl |
| I′-180 | Ru | II′-5 | —Cl |
| I′-181 | Ru | II′-6 | —Cl |
| I′-182 | Ru | II′-25 | —Cl |
| I′-183 | Ru | II′-26 | —Cl |
| I′-184 | Ru | II′-27 | —Cl |
| I′-185 | Ru | II′-28 | —Cl |
| I′-186 | Ru | II′-29 | —Cl |
| I′-187 | Ru | II′-31 | —Cl |
| I′-188 | Ru | II′-32 | —Cl |
| I′-189 | Ru | II′-34 | —Cl |
| I′-190 | Ru | II′-35 | —Cl |
| I′-191 | Ru | II′-37 | —Cl |
| I′-192 | Ru | II′-43 | —Cl |
| I′-193 | Ru | II′-44 | —Cl |
| I′-194 | Ru | II′-45 | —Cl |
| I′-195 | Ru | II′-46 | —Cl |
| I′-196 | Ru | II′-48 | —Cl |
| I′-197 | Ru | II′-49 | —Cl |
| I′-198 | Ru | II′-53 | —Cl |
| I′-199 | Ru | II′-54 | —Cl |
| I′-200 | Ru | II′-55 | —Cl |

TABLE 4-6

| Compound | M′ | Compound (II′) | X₁ = X₂ |
|---|---|---|---|
| I′-201 | Ru | II′-59 | —Cl |
| I′-202 | Ru | II′-60 | —Cl |
| I′-203 | Ru | II′-61 | —Cl |
| I′-204 | Ru | II′-75 | —Cl |
| I′-205 | Os | II′-1 | —NCS |
| I′-206 | Os | II′-2 | —NCS |
| I′-207 | Os | II′-5 | —NCS |
| I′-208 | Os | II′-6 | —NCS |
| I′-209 | Os | II′-25 | —NCS |
| I′-210 | Os | II′-26 | —NCS |
| I′-211 | Os | II′-27 | —NCS |
| I′-212 | Os | II′-28 | —NCS |
| I′-213 | Os | II′-29 | —NCS |
| I′-214 | Os | II′-31 | —NCS |
| I′-215 | Os | II′-32 | —NCS |
| I′-216 | Os | II′-34 | —NCS |
| I′-217 | Os | II′-35 | —NCS |
| I′-218 | Os | II′-37 | —NCS |
| I′-219 | Os | II′-43 | —NCS |
| I′-220 | Os | II′-44 | —NCS |
| I′-221 | Os | II′-45 | —NCS |
| I′-222 | Os | II′-46 | —NCS |
| I′-223 | Os | II′-48 | —NCS |
| I′-224 | Os | II′-49 | —NCS |
| I′-225 | Os | II′-53 | —NCS |
| I′-226 | Os | II′-54 | —NCS |
| I′-227 | Os | II′-55 | —NCS |
| I′-228 | Os | II′-59 | —NCS |
| I′-229 | Os | II′-60 | —NCS |
| I′-230 | Os | II′-61 | —NCS |
| I′-231 | Os | II′-75 | —NCS |
| I′-232 | Fe | II′-1 | —NCS |
| I′-233 | Fe | II′-2 | —NCS |
| I′-234 | Fe | II′-5 | —NCS |
| I′-235 | Fe | II′-6 | —NCS |
| I′-236 | Fe | II′-25 | —NCS |
| I′-237 | Fe | II′-26 | —NCS |
| I′-238 | Fe | II′-27 | —NCS |
| I′-239 | Fe | II′-28 | —NCS |
| I′-240 | Fe | II′-29 | —NCS |

TABLE 4-7

| Compound | M′ | Compound (II′) | X₁ = X₂ |
|---|---|---|---|
| I′-241 | Fe | II′-31 | —NCS |
| I′-242 | Fe | II′-32 | —NCS |
| I′-243 | Fe | II′-34 | —NCS |
| I′-244 | Fe | II′-35 | —NCS |
| I′-245 | Fe | II′-37 | —NCS |
| I′-246 | Fe | II′-43 | —NCS |
| I′-247 | Fe | II′-44 | —NCS |
| I′-248 | Fe | II′-45 | —NCS |
| I′-249 | Fe | II′-46 | —NCS |
| I′-250 | Fe | II′-48 | —NCS |
| I′-251 | Fe | II′-49 | —NCS |
| I′-252 | Fe | II′-53 | —NCS |
| I′-253 | Fe | II′-54 | —NCS |
| I′-254 | Fe | II′-55 | —NCS |
| I′-255 | Fe | II′-59 | —NCS |
| I′-256 | Fe | II′-60 | —NCS |
| I′-257 | Fe | II′-61 | —NCS |
| I′-258 | Fe | II′-75 | —NCS |

The compounds (II) and (II′), and the complexes (I) and (I′) can usually be identified by using such means as NMR, LC-MS and the like.

The photosensitizing dye of the present invention is a dye comprising the complex compound (I) or (I′). The dye may contain complex compound (I) or (I′) only or may obtain compounds of different kinds from complex compound (I) or (I′).

The dyes which may be mixed with complex compounds (I) and (I′) include metal complexes, organic dyes and the like, which have absorptions at a wavelength around 300 to 700 nm.

Specific examples of metal complexes which may be mixed include metal phthalocyanines such as copper phthalocyanine and titanyl phthalocyanine; chlorophill and hemin; and ruthenium, osmium, iron and zinc complexes described in Japanese Patent Laid-Open No. Hei-1-220380 and National Publication of International Patent Application No. Hei-5-504023.

More detailed examples of the ruthenium complexes include cis-bis(isothiocyanato)bis(2,2′-bipyridyl-4,4′-dicarboxylato)-ruthenium(II)bis-tetrabutylammonium, cis-bis(isothiocyanato)bis(2,2′-bipyridyl-4,4′-dicarboxylato)-ruthenium(II), tris(isothiocyanato)-ruthenium(II)-2,2′:6′,2″-terpyridine-4,4′,4″-tricarboxylic acid-tris-tetrabutylammonium, and cis-bis(isothiocyanato)(2,2′-bipyridyl-4,4′-dicarboxylato)(2,2′-bipyridyl-4,4′-dinonyl)-ruthenium(II).

Organic dyes include, for example, metal free phthalocyanines, cyanine dyes, melocyanine dyes, xanthene dyes, triphenylmethane dyes, coumarin dyes, organic dyes such as indolines, and squalilium dyes.

The cyanine dyes include, specifically, NK1194, NK3422 (both manufactured by Nippon Kankoh-Shikiso Kenkyusho Co., Ltd.) and the like. The melocyanine dyes include, specifically, NK2426 and NK2501 (both manufactured by Nippon Kankoh-Shikiso Kenkyusho Co., Ltd.).

The xanthene dyes include, for example, uranine, eosin, rose bengal, rhodamine B and dibromofluorescein.

The triphenylmethane dyes include, for example, malachite green and crystal violet.

The coumarin dyes include compounds having a structural unit shown below, such as NKX-2677 (manufactured by Hayashibara Biochemical Laboratories, Inc.).

The indoline dyes are exemplified by compounds having a structural unit shown below, such as D149 (manufactured by Mitsubishi Paper Mills Limited).

The squalilium dyes are exemplified specifically by compounds having a structural unit shown below.

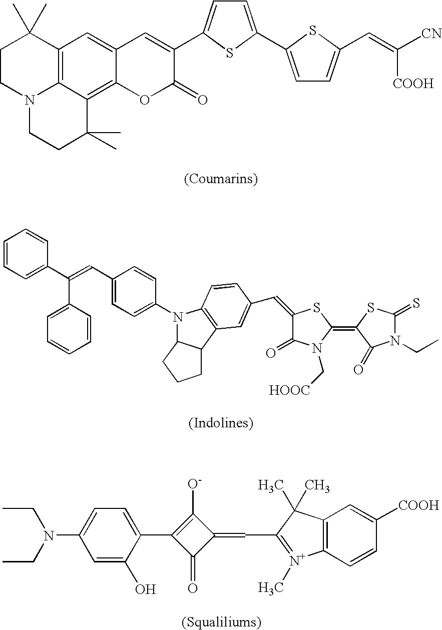

The photoelectrochemical cell of the present invention comprises a photoelectric converter, a charge transport layer and a counter electrode, and can convert light into electricity. In a photoelectrochemical cell, there are laminated sequentially a photoelectric converter, a charge transport layer and a counter electrode. And, when the electroconductive substrate of the photoelectric converter is connected with the counter electrode, the charge is transferred, namely, electricity is generated.

As other photoelectrochemical cells, there may be exemplified, for example, a photoelectrochemical cell which comprises a plurality of laminated portions comprising a photoelectric converter and a charge transport layer, and one counter electrode; and, for example, a photoelectrochemical cell which comprises a plurality of photoelectric converters, one charge transport layer and one counter electrode.

The photoelectrochemical cells are classified broadly into a wet-type photoelectrochemical cell and a dry-type photoelectrochemical cell. In the wet-type photoelectrochemical cell, the charge transport layer contained is a layer composed of an electrolytic solution and, usually, as the charge transport layer, there is filled an electrolytic solution between the photoelectric converter and the counter electrode.

The dry-type photoelectrochemical cell includes, for example, a cell wherein the charge transport layer between the photoelectric converter and the counter electrode comprises a solid hole-transport material.

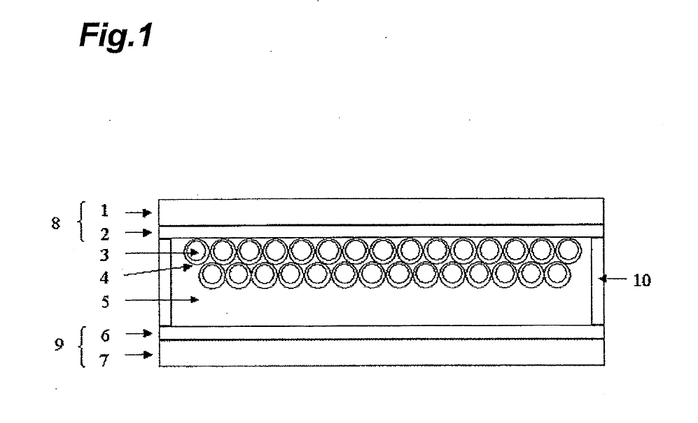

One embodiment of the photoelectrochemical cells is shown in FIG. 1. There exist a conductive substrate 8, a counter electrode (conductive substrate) 9 facing the conductive substrate 8, and, between these, a semiconductor fine particles layer 3 on which dyes 4 for a photoelectric converter are adsorbed. When making a wet-type photoelectric converter, the semiconductor particles layer 3 is filled with an electrolytic solution 5 and sealed with a sealant 10.

Here, the primary particle size of the semiconductor fine particles used for the photoelectric converter is usually about 1 to 5,000 nm, preferably about 5 to 300 nm. With an aim to improve photoelectric conversion efficiency by reflection, semiconductor fine particles of different primary particle size may be mixed. In addition, tube- or hollow-shaped fine particles may be used.

Here, the primary particle size of the semiconductor fine particles used for the photoelectric converter is usually about 1 to 5,000 nm, preferably about 5 to 300 nm. With an aim to improve photoelectric conversion efficiency by reflection, semiconductor particles of different primary particle size may be mixed. In addition, tube- or hollow-shaped fine particles may be used.

The material compound which constitute the semiconductor fine particles include, for example, metal oxides such as titanium oxide, tin oxide, zinc oxide, iron oxide, tungsten oxide, zirconium oxide, hafnium oxide, strontium oxide, indium oxide, cerium oxide, yttrium oxide, lanthanum oxide, vanadium oxide, niobium oxide, tantalum oxide, gallium oxide, nickel oxide, strontium titanate, barium titanate, potassium niobate, and sodium tantalate;

metal halides such as silver iodide, silver bromide, copper iodide, and copper bromide;

metal sulfides such as zinc sulfide, indium sulfide, bismuth sulfide, cadmium sulfide, zirconium sulfide, tantalum sulfide, molybdenum sulfide, silver sulfide, copper sulfide, tin sulfide, tungsten sulfide and antimony sulfide;

metal selenides such as cadmium selenide, zirconium selenide, zinc selenide, titanium selenide, indium selenide, tungsten selenide, molybdenum selenide, bismuth selenide and lead selenide;

metal tellurides such as cadmium telluride, tungsten telluride, molybdenum telluride, zinc telluride and bismuth telluride;

metal phosphides such as zinc phosphide, gallium phosphide, indium phosphide and cadmium phosphide; and

material compounds such as gallium arsenide, copper-indium selenide, copper-indium sulfide, silicon, and germanium.

Further, there may be used mixtures of two or more kinds of material compounds such as zinc oxide/tin oxide and tin oxide/titanium oxide.

Above all, metal oxides such as titanium oxide, tin oxide, zinc oxide, iron oxide, tungsten oxide, zirconium oxide, hafnium oxide, strontium oxide, indium oxide, cerium oxide, yttrium oxide, lanthanum oxide, vanadium oxide, niobium oxide, tantalum oxide, gallium oxide, nickel oxide, strontium titanate, barium titanate, potassium niobate, sodium tantalate, zinc oxide/tin oxide, tin oxide/titanium oxide are preferable because they are relatively moderately priced, easy to acquire and easily dyed with pigments. Especially, titanium oxide is preferable.

As the conductive substrates (8 and 9 in FIG. 1) used for the photoelectric converter, there may be used a conductive material itself or a substrate on which a conductive material is overlaid. The conductive materials include a metal such as platinum, gold, silver, copper, aluminum, rhodium, indium, titanium, palladium or iron; an alloy of the metals; a conductive metal oxide such as indium-tin multiple oxide or tin oxide doped with fluorine; a carbon; a conductive polymer such as polyethylenedioxythiophene (PEDOT) and polyaniline. The conductive polymer may be doped, for example, with para-toluenesulfonic acid.

In order to confine the incoming light and utilize it efficiently, the conductive substrate is preferably one which has a texture configuration on its surface. As for the conductive layers (2 and 6 in FIG. 1), the lower the resistance, the better. Also, it has preferably high transparency (transmittance is 80% or more at a longer wavelength than 350 nm).

As the conductive substrates (8 and 9 in FIG. 1), preferable is one where a conductive metal oxide is coated on glass or plastic. Above all, especially preferable is conductive glass on which is laminated a conductive layer comprising tin dioxide doped with fluorine. When a plastic substrate is employed, there may be used polyethylene terephthalate (PET), polyethylene naphthalate (PEN), polyphenylene sulfide (PPS), polycarbonate (PC), polypropylene (PP), polyimide (PI), triacetyl cellulose (TAC), syndiotactic polystyrene (SPS), polyarylate (PAR); cyclic polyolefins (COP) such as ARTON (a registered trademark of JSR Corporation), ZEONOR (a registered trademark of Nippon Zeon Co., Ltd.), APEL (a registered trademark of Mitsui Chemicals, Inc.); and Topas (a registered trademark of Ticona Inc.); polyethersulfone (PES), polyetherimide (PEI), polysulfone (PSF), polyamide (PA) and the like.

Among these, especially preferable is conductive PET due to its low resistance, good transparency and easy availability, the conductive PET comprising a deposited conductive layer, which comprises an indium-tin multiple oxide.

Methods for forming a layer of semiconductor fine particles on a conductive substrate are exemplified by a method whereby a thin film of semiconductor fine particles is directly formed on the conductive substrate by spray atomization or the like; a method where a thin film of semiconductor fine particles is electrically made to precipitate using the conductive substrate as an electrode; and a method whereby a slurry of semiconductor fine particles is coated on a conductive substrate and, thereafter, dried, cured or burned to produce a semiconductor fine particles layer.

The method to coat a slurry of semiconductor fine particles on a conductive substrate includes means such as, for example, doctor blade, squeegee, spin coat, dip coat and screen printing. In the case of this method, the average particle size of the semiconductor fine particles in a dispersed state in the slurry is preferably 0.01 μm to 100 μm. The dispersion medium to disperse the slurry may be any as long as it can disperse the semiconductor fine particles. There may be used water or organic solvents, including alcohol solvents such as ethanol, isopropanol, t-butanol and terpineol; ketone solvents such as acetone; and the like. These water and organic solvents may be mixtures. In the dispersion liquid, there may be contained polymers such as polyethylene glycols; surface active agents such as Triton-X; organic acids or inorganic acids such as acetic acid, formic acid, nitric acid and hydrochloric acid; chelating agents such as acetyl acetone.

The conductive substrate coated with the slurry is burnt. The burning temperature is below the melting temperature (or the softening temperature) of the substrate including thermoplastic resin and the like, and, usually, the upper limit of the burning temperature is 900° C., preferably 600° C. or lower. In addition, the burning time is usually 10 hours or less. The thickness of the semiconductor fine particles layer on the conductive substrate is usually 1 to 200 μm, preferably 5 to 50 μm.

As methods for forming a layer of semiconductor fine particles on a conductive substrate at relatively low temperature, there may mentioned a hydrothermal method whereby a porous layer of semiconductor fine particles is formed by a hydrothermal treatment (Hideki Minoura in “Dye-Sensitized Photoelectrochemical Cell toward the Practical Use”; NTS Inc., 2003; Lecture 2, pp. 63-65), a electrophoretic deposition method whereby a dispersion liquid of semiconductor particles dispersed is electrodeposited on a substrate (T. Miyasaka et al., Chem. Lett., 1259 (2002); a press method whereby a semiconductor paste is coated on a substrate, dried and, thereafter, pressed (Takehiko Yorozu in “Dye-Sensitized Photoelecrochemical Cell toward the Practical Use”; NTS Inc., 2003; Lecture 12, pp 312-313) and the like.

On the surface of the semiconductor fine particles layer, there may be provided a chemical plating treatment using an aqueous solution of titanium tetrachloride or an electrochemical plating treatment using an aqueous solution of titanium trichloride. By these treatments, it becomes possible to increase the surface area of the semiconductor fine particles, to improve purity of the neighborhood of the semiconductor fine particle, to envelope impurities present on the surface of the semiconductor fine particles such as iron or to improve the connecting and binding properties of the semiconductor fine particles.

The semiconductor fine particles are preferably those having a large surface area so that they can adsorb many photosensitizing dyes. For this purpose, the surface area of the semiconductor fine particles layer in a state of being coated on the substrate is, preferably, 10 times or more as large as the projected area, more preferably 100 times or more as large. The maximum of this value is usually about 1,000 times.

The layer of the semiconductor fine particles is not limited to a single layer with a thickness of one fine particle but may contain a plurality of layers piled up, each layer comprising particles of different diameters.

As a method to have the photosensitizing dyes of the present invention adsorbed on the semiconductor fine particles, there is used a method whereby well-dried semiconductor fine particles are dipped, for about 1 minute to 24 hours, in a solution of the photosensitizing dye of the present invention. Adsorption of the photosensitizing dye may be carried out either at room temperature or under heating to reflux. Adsorption of the photosensitizing dye may be carried out before coating of the semiconductor fine particles or after the coating. Or adsorption may be carried out by coating the semiconductor particles and photosensitizing dyes at the same time. However, it is more preferable to have the photosensitizing dye adsorbed on the semiconductor fine particles film after the latter is coated. When the semiconductor fine particles layer is subjected to a heat treatment, adsorption of the photosensitizing dye is preferably carried out after the heat treatment. Especially preferable is a method whereby the photosensitizing dye is adsorbed quickly after the heat treatment, before moisture is adsorbed on the surface of the fine particles layer.

In order to prevent decrease in a sensitizing effect due to flotation of the photosensitizing dye which has not been adsorbed on the semiconductor fine particles, it is desirable to remove the unadsorbed photosensitizing dyes by washing.

The photosensitizing dye to be adsorbed may be of one kind or may be used as a mixture of several kinds. When the application is a photoelectrochemical cell, it is preferable to select the photosensitizing dyes to be mixed so that the wavelength range, where irradiated light such as sunlight is photoelectrically converted, is made as wide as possible. In addition, the amount of the photosensitizing dye to be adsorbed on the semiconductor fine particles is preferably 0.01 to 1 millimole per 1 g of the semiconductor fine particles. The amount of the dye in this range is preferable because a sensitizing effect on the semiconductor fine particles is obtained sufficiently and there is a tendency that decrease in the sensitizing effect is prevented, the decrease being due to flotation of the photosensitizing dye not adhered to the semiconductor fine particles.

In order to prevent the photosensitizing dyes from mutual interaction such as association and agglomeration among themselves, there may be coadsorbed colorless compounds. The colorless compounds to be coadsorbed are preferably colorless hydrophobic compounds. The hydrophobic compounds include steroid compounds having carboxyl groups (for example, chenodeoxycholic acid) and the like. Further, for the purpose of facilitating removal of the excessive photosensitizing dyes, the surface of the semiconductor fine particles may be treated with amines after adsorption of the dyes. Preferable amines include pyridine, 4-tert-butylpyridine, polyvinylpyridine and the like. When these are liquids, these may be used as they are; when they are solids, they may be used dissolved in organic solvents.

The aforementioned conductive substrate 8 comprises, in order from the top, substrate 1 and conductive layer 2. Counter electrode 9 comprises, in order from the bottom, substrate 7 and conductive layer 6.

When the photoelectrochemical cell of the present invention is a wet-type photoelectrochemical cell, the electrolyte which is used in the electrolytic solution contained in the wet-type photoelectrochemical cell includes, for example, a combination of I₂ and various iodides, a combination of Br₂ and various bromides, a combination of metal complexes of a ferrocyanic acid salt/a ferricyanic acid salt, a combination of metal complexes of ferrocene/a ferricinium ion, a combination of sulfur compounds of an alkylthiol/an alkyl disulfide, a combination of an alkylviologen and a reduced form thereof, and a combination of polyhydroxybenzenes and oxidized forms thereof.

Here, the iodides which may be combined with I₂ include, for example, metal iodides such as LiI, NaI, KI, CsI and CaI₂; iodide salts of quaternary imidazolium compounds such as 1-propyl-3-methylimidazolium iodide and 1-propyl 2,3-dimethylimidazolium iodide; iodide salts of quaternary pyridinium compounds; iodide salts of tetraalkylammonium compounds.

The bromides which may be combined with Br₂ include, for example, metal bromides such as LiBr, NaBr, KBr, CsBr and CaBr₂; bromide salts of quaternary ammonium compounds such as tetraalkylammonium bromides and pyridinium bromides.

The alkyl viologens include, for example, methylviologen chloride, hexylviologen bromide and benzylviologen tetrafluoroborate. Polyhydroxybenzenes include, for example, hydroquinone and naphthohydroquinone. As the electrolyte, preferable above all is a combination of at least one iodide compound and I₂, the iodide compound selected from the group consisting of metal iodides, quaternary imidazolium iodides, quaternary pyridinium iodides and tetraalkylammonium iodides.

The organic solvents used for the above-described electrolytic solution include nitrile solvents such as acetonitrile, methoxyacetonitrile and propionitrile;

carbonate solvents such as ethylene carbonate and propylene carbonate;

1-methyl-3-propylimidazolium iodide and 1 -methyl-3-hexylimidazolium iodide;

ionic liquids such as 1-ethyl-3-methylimidazolium bis(trifluoromethanesulfonyl)imide. Further, lactone solvents such as γ-butyrolactone; and amide solvents such as N,N-dimethylformamide may be mentioned. These solvents may be gelled by polyacrylonitrile, polyvinylidene fluoride, poly-4-vinylpyridine and a low-molecular gelling agent such as the one shown in Chemistry Letters, 1241 (1998).

In the photoelectrochemical cell of the present invention, a solid hole-transport material may be used instead of the electrolytic solution.

As the hole-transport materials, there may be mentioned p-type inorganic semiconductors containing univalent copper such as CuI and CuSCN, and conductive polymers including such arylamines as shown by Synthetic Metal, 89, 215 (1997) and Nature, 395, 583 (1998); polythiophene and derivatives thereof; polypyrrole and derivatives thereof; polyaniline and derivatives thereof; poly(p-phenylene) and derivatives thereof; poly(phenylenevinylene) and derivatives thereof; and the like.

The counter electrode, which constitutes the photoelectric converter of the present invention, is an electrode having conductivity and, in order to maintain strength and to improve airtightness, there may be used the same substrate as the aforementioned conductive substrate.

In order for the light to reach the semiconductor fine particles layer on which are adsorbed the photosensitizing dyes, usually, at least either of the conductive substrate and counter electrode is practically transparent. In the photoelectric converter of the present invention, preferable is the one where the conductive substrate having a layer of semiconductor fine particles is transparent and irradiated light is made incident from the side of the conductive substrate. In this case, it is further preferable that the counter electrode 9 has a property to reflect light.

As the counter electrode 9 of the photoelectric converter, there may be used, for example, glass or plastic on which a metal, carbon or a conductive oxide is vapor-deposited. Further, the counter electrode may be prepared by forming a conductive layer by vapor deposition or sputtering so that the thickness thereof falls in a range of 1 mm or less, preferably in a range of 5 nm to 100 μm. In the present invention, it is preferable to use, glass on which platinum or carbon is vapor-deposited or to use a counter electrode on which a conductive layer is formed by vapor deposition or sputtering.

In order to prevent leakage or evaporation of the electrolytic solution, the photoelectrochemical cell may be sealed by using a sealant. As the sealant, there may be used ionomer resins such as Himilan (manufactured by Mitsui-DuPont Polychemical Co., Ltd.); glass frit; hot-melt adhesives such as SX1170 (manufactured by Solaronix SA); adhesives such as Amosil 4 (manufactured by Solaronix SA); and BYNEL (manufactured by duPont Company).

In the following, the present invention will be described in more detail by referring to Examples and the like but the present invention is not limited by these Examples.

### Example 1

### Manufacturing Example 1

### Manufacturing Example of Complex Compound (I-16)

Q-1 (1.95 g, 7.33 mmol) was dissolved in 55 g of 1,2-dichloroethane, followed by addition of manganese dioxide (4.29 g, 37.1 mmol) and reflux for 3 hours. After the reaction, the reaction mixture was filtered through celite and the filter cake was washed with chloroform. The filtrate was concentrated to obtain 1.03 g (yield, 49%) of Q-2 of 93.4% purity by HPLC. Then, to Q-3 (0.90 g, 1.77 mmol) was added 8.9 g of tetrahydrofuran and the mixture was ice-chilled. An n-butyllithium/hexane solution (0.5 ml, 0.80 mmol) was added dropwise over a ca. 10 minute period and the mixture was allowed to react for 1 hour at the same temperature. Thereto, a solution of Q-2 (0.90 g, 3.42 mmol) in 1 ml of tetrahydrofuran was dropwise added over a ca. 5 minute period

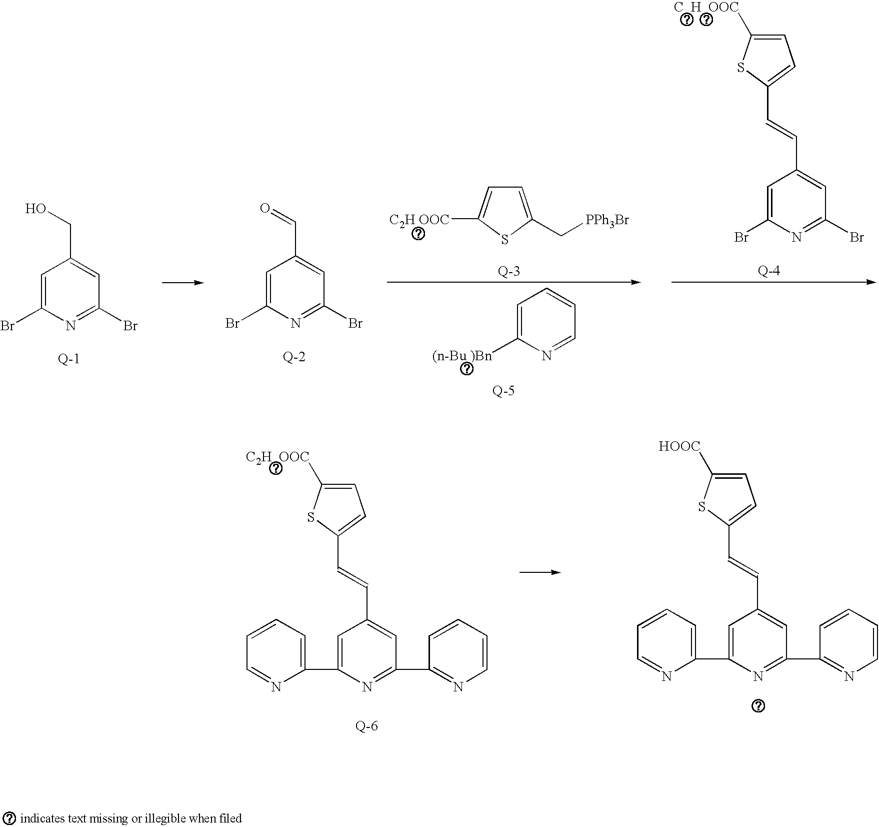

and was allowed to react at the same temperature for 2 hours, followed by warming to room temperature and stirring for 2 hours. After the reaction, the solvent was distilled off under reduced pressure. To the residue obtained was poured water and extracted with chloroform. The chloroform layer was washed with water and dried over magnesium sulfate. The solvent was distilled off under reduced pressure and the residue obtained was purified by column chromatography to obtain 0.29 g (yield, 31%) of Q-4 of 80.5% purity by HPLC.

To the obtained Q-4 (0.32 g, 0.77 mmol) were added Q-5 (0.39 g, 1.08 mmol), Pd(PPh₃)₄ (88 mg, 0.08 mmol) and 3.2 g of toluene, and the mixture was refluxed for 4 hours. After the reaction, the solvent was distilled off under reduced pressure and the reside was purified by column chromatography to obtain 0.38 g (yield, 84%) of Q-6 of 71.9% purity by HPLC.

Subsequently, the obtained Q-6 (0.28 g, 0.67 mmol) was dissolved in 5 ml of ethanol and, thereto, lithium hydroxide (0.48 g, 20.02 mmol) and 2 ml of water were added, and the mixture was refluxed for 2 hours to carry out hydrolysis of the carboxylic acid ester. After confirming completion of the reaction, the reaction mixture was neutralized with 2N hydrochloric acid. Water was removed by codistillation with ethanol to obtain 11-16. The solid material obtained was confirmed to be the desired compound (11-16, mw 385) by ESI-MS.

Compound (II-16) ESI-MS (m/z)

m/z=386 [M+H]+

To 23 mg (0.06 mmol) of II-16 obtained was added ethanol and, further, there was charged 18 mg (0.07 mmol) of ruthenium chloride trihydrate. The reaction mixture was stirred for 3 hours under a refluxing condition and, after being allowed to cool, the reaction mixture was concentrated under reduced pressure to obtain dark violet-colored crystals. The crystals obtained were dissolved in 10 ml of DMF, potassium thiocyanate (119 mg, 1.34 mmol) and 1 ml of water were added thereto, and the reaction mixture was stirred under heating at 150° C. for 4 hours. The reaction liquid was concentrated by means of an evaporator and, from the concentrated residue, the main component was fractionated by high-speed liquid chromatography to obtain a violet solid. The solid obtained was identified as the desired compound (I-16, mw 660) by ESI-MS.

Complex compound (I-16) ESI-MS (m/z)

m/z=661 [M+H]+

### <Preparation of a Photoelectrochemical Cell Comprising Complex Compound (I-16)>

On a conductive surface of a conductive substrate, conductive glass provided with a tin oxide film doped with fluorine (manufactured by Nippon Sheet Glass Co., Ltd., 10Ω/□), a dispersion liquid of titanium oxide, Ti-Nanoxide T/SP (trade name, manufactured by Solaronix SA), was coated by means of a screen printer, thereafter burned at 500° C. and the glass was cooled to have a layer of semiconductor particles laminated on a conductive substrate. Subsequently, the glass was dipped for 16 hours in a solution of compound (I-16) (the concentration, 0.0003 mol/l; solvent, N,N-dimethylacetamide; 0.03 ml/l of chenodeoxycholic acid (hereinafter abbreviated as DCA) was added). After taking the glass out of the solution, it was washed with acetonitrile and dried naturally to obtain a laminated body (the area of the titanium oxide electrode was 24 mm²) comprising a conductive substrate and a layer of semiconductor fine particles on which photosensitizing dyes are adsorbed. Then, after disposing a 25 μm-thick polyethylene terephthalate film around the layer as a spacer, the layer was impregnated with an electrolytic solution (solvent, acetonitrile; iodine concentration in the solvent, 0.05 mol/l; lithium iodide concentration in the solvent, 0.1 mol/l; 4-t-butylpyridine concentration in the solvent, 0.5 mol/l; 1-propyl-2,3-dimethylimidazolium iodide concentration in the solvent, 0.6 mol/l). Finally, the counter electrode, glass vapor-deposited with platinum, was superposed to obtain a photoelectrochemical cell comprising a conductive substrate, a layer of semiconductor fine particles on which photosensitive dyes are adsorbed and a counter electrode of the conductive substrate, with an electrolytic solution impregnated between the conductive substrate and the counter electrode. With thus prepared photoelectrochemical cell, using an IPCE (incident photon-to-current efficiency) measuring instrument. (manufactured by Bunko Keiki Co., Ltd), the IPCE was measured. The results are shown in Table 5.

### Example 2

### Manufacturing Example 2

### Manufacturing Example of Complex Compound (I-30)

To Q-8 (0.70 g, 2.07 mmol), obtained in the same manner as in Manufacturing Method 1 except that the reaction was carried out using Q-7 instead of Q-2, and a tin reagent XI-1 (1.29 ml, 6.21 mmol) and PdCl₂(PPh₃)₂ (0.29 g, 0.41 mmol) were dissolved in 120 ml of 1,2-dimethoxyethane and the solution was refluxed for 1 hour. After the reaction, the solvent was distilled off under reduced pressure and the residue was dissolved in diethyl ether. The insoluble matter was removed by filtration, and from the filtrate, the solvent was distilled off to obtain tin compound Q-9. Then, to Q-9 obtained were added Q-10 (0.26 g, 1.03 mmol), PdCl₂(PPh₃)₂ (0.29 g, 0.41 mmol) and 5 ml of toluene and the mixture was refluxed for 11 hours. After the reaction,

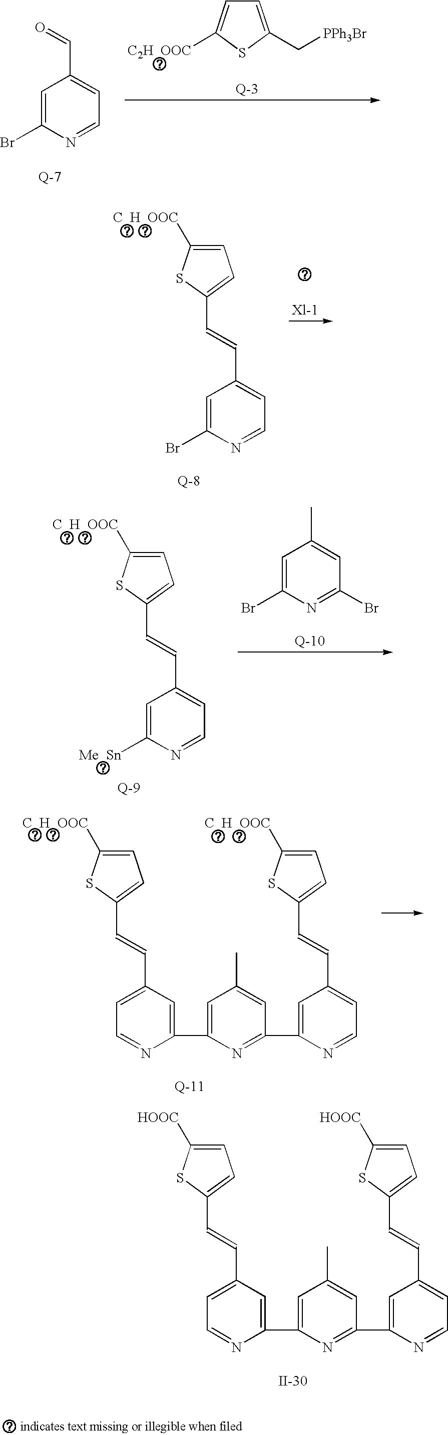

the solvent was distilled off under reduced pressure and the residue was purified by column chromatography to obtain 0.16 g (yield, 21%) of Q-11 of 81.6% purity by HPLC.

Subsequently, the obtained Q-11 (56 mg, 0.10 mmol) was dissolved in 5 ml of ethanol, lithium hydroxide (47 mg, 0.20 mmol) and 1 ml of water were added thereto and the mixture was refluxed for 2 hours. After completion of the reaction was confirmed, the reaction mixture was neutralized with 2N hydrochloric acid and water was removed by codistillation with ethanol to obtain II-30. The solid material obtained was confirmed to be the desired compound (II-30, mw 551) by ESI-MS.

Compound (II-30) ESI-MS (m/z)

m/z =552 [M+H]+

Using 11-30 obtained, a reaction was carried out in the same manner as in Example 1 to obtain 1-30.

### Example 3

### Manufacturing Example 3

### Manufacturing Example of Complex Compound (I-25)

Q-12 (0.32 g, 1.23 mmol), XI-1 (0.16 ml, 0.49 mmol) and Pd(PPh₃)₄ (54 mg, 0.05 mmol) were dissolved in 5 ml of 1,2-dimethoxyethane and the solution was refluxed for 1 hour. After the reaction, the solvent was distilled off under reduced pressure and the residue was dissolved in diethyl ether. The insoluble matter was removed by filtration, and from the filtrate, the solvent was distilled off to obtain Q-13.

Then, to Q-13 obtained was added Q-8 (0.13 g, 0.39 mmol), PdCl₂(PPh₃)₂ (47 mg, 0.07 mmol) and 5 ml of toluene, and the mixture was refluxed for 11 hours. After the reaction, the solvent was distilled off under reduced pressure and

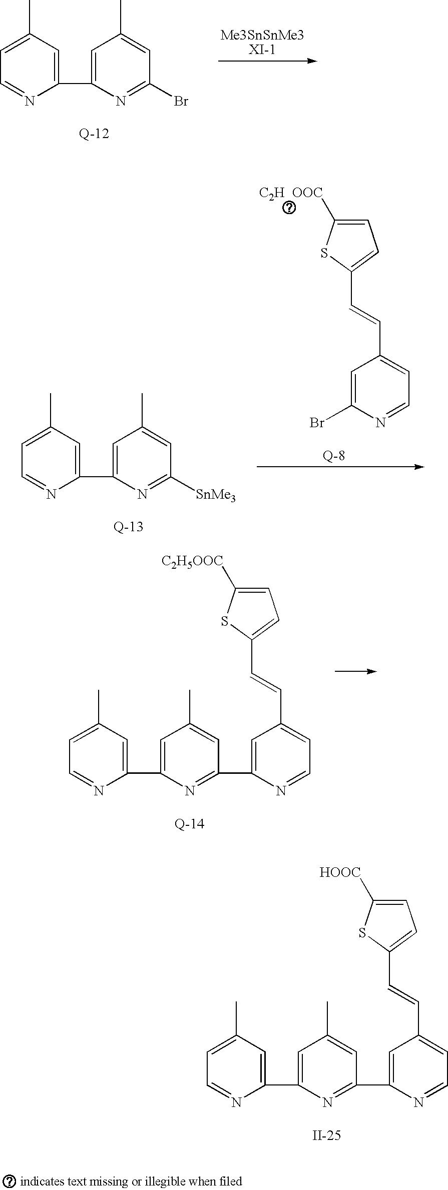

the residue was purified by column chromatography to obtain 0.23 g (yield, 83%) of Q-14 of 65.7% purity by HPLC.

Subsequently, the obtained Q-14 was hydrolyzed in the same manner as in Manufacturing Method 1 to obtain II-25. The solid material obtained was confirmed to be the desired compound (II-25, mw 413) by ESI-MS.

Compound (11-25) ESI-MS (m/z)

m/z=414 [M+H]+

Using II-25 obtained, a reaction was carried out in the same manner as in Example 1 to obtain I-25.

Complex compound (I-25) ESI-MS (m/z)

m/z =689 [M+H]+

With complex compound (I-25) obtained in Example 3 also, there was measured IPCE in the same manner as in Example 1. The IPCE of the photoelectric converter obtained in Example 3 is shown in Table 6.

### Comparative Examples 1 and 2

Except that cis-bis(isothiocyanato)bis(2,2′-bipyridyl-4,4′-dicarboxylate)-ruthenium(II)bis-tetrabutylammonium (hereinafter abbreviated as complex compound (2)) was used as the photosensitizing dye and that t-butanol/acetonitrile=1/1 (vol/vol) was used as the dissolving solvent, a cell was prepared in the same manner as in Example 1 to obtain a photoelectrochemical cell. Then, the IPCE was measured in the same manner as in Example 1. The results are described in Table 5 and Table 6. In addition, the cells in Comparative Examples 1 and 2 were prepared and evaluated on the same day as those of the compounds of the Examples described in the same Tables.

TABLE 5

| | | Comparative |
|---|---|---|
| | Example 1 | Example 1 |
| Complex | (I-16) | (2) |
| compound | | |
| Amount of DCA | 0.03 | 0 |
| added (mol/L) | | |
| IPCE (750 nm) | 34.1% | 8.1% |
| IPCE (800 nm) | 11.6% | 1.2% |

TABLE 6

| | | Comparative |
|---|---|---|
| | Example 3 | Example 2 |
| Complex | (I-25) | (2) |
| compound | | |
| Amount of DCA | 0.12 | 0 |
| added (mol/L) | | |
| IPCE (750 nm) | 15.6% | 8.3% |
| IPCE (800 nm) | 3.4% | 1.2% |

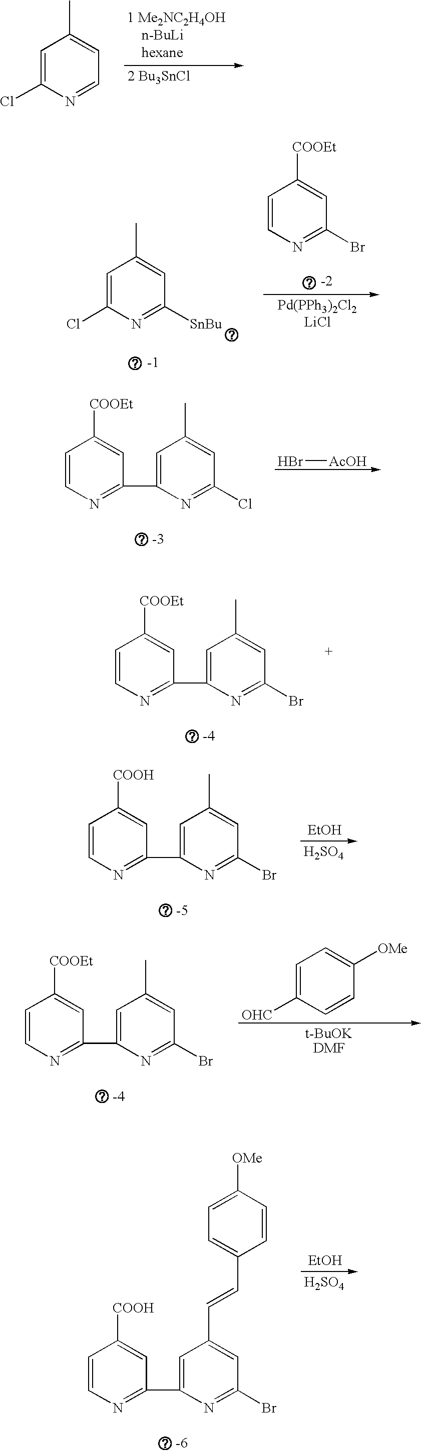

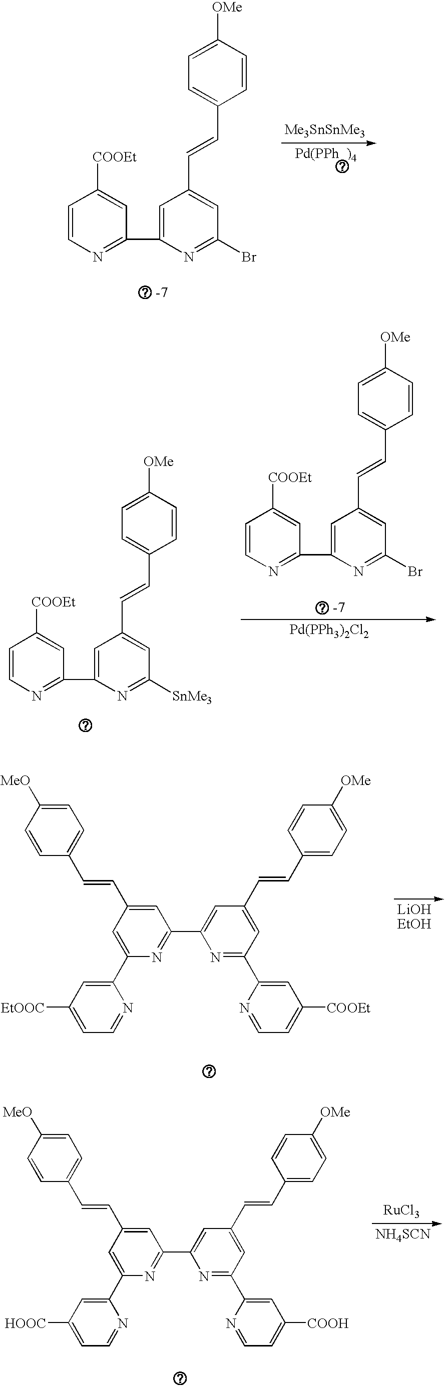

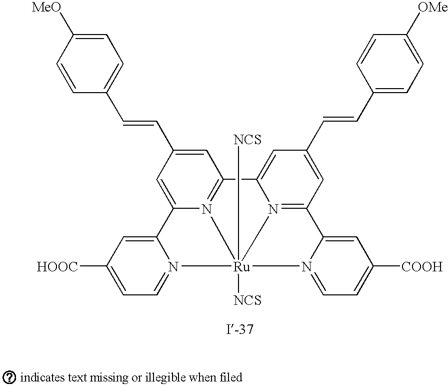

### Example 4

### Manufacturing Example 4

### Manufacturing Example of Complex Compound (I′-37)

### Synthesis of Compound (B-1)

To a 2 l four-necked flask were charged 250 ml of n-hexane and 50.6 g (0.57 mol) of dimethylethanolamine, and the mixture was cooled to −30° C. Under a nitrogen atmosphere, 710 ml (1.14 mol) of a hexane solution of n-BuLi (1.6 mol/L) was added dropwise at inner temperature in a range of −10° C. to −5° C. After the dropwise addition, the reaction mixture was stirred at a temperature in a range of −20° C. to −10° C. for 30 minutes. The mixture was cooled to inner temperature of −40° C. and 36.5 g (0.29 mol) of 2-chloropicoline was dropwise added thereto at inner temperature in a range of −40° C. to −20° C., followed by subsequent stirring at temperature in a range of −40° C. to −30° C. for 1 hour.

The reaction mixture was cooled to −70° C. and, at inner temperature below −30° C., 111.0 g (0.34 mol) of n-Bu₃SnCl was dropwise added thereto, followed by stirring overnight. The reaction mixture was cooled to inner temperature of −5° C. and 500 ml of deionized water was dropwise added at inner temperature of 5° C. or below. The aqueous layer was extracted with 400 ml of ethyl acetate. The organic layers were combined, washed with 1,500 ml of saturated saline, dried over sodium sulfate and concentrated under reduced pressure. The residue was purified by silica column chromatography (n-hexane:ethyl acetate:triethylamine=200:10:20) to obtain 66.0 g (yield, 55% (the yield is an apparent yield (hereinafter, the same shall apply); HPLC purity, 89.5%) of the desired compound (B-1).

Compound (B-1) ESI-MS (m/z) m/z=417.1 [M+H]+

### Synthesis of Compound (B-3)

To a 100 ml two-necked flask, there were added successively 30 ml of anhydrous toluene, 5.7 g (25 mmol) of compound (B-2), 12.4 g (30 mmol) of compound (B-1), 1.0 g (24 mmol) of LiCl and 6.1 mg (8.7 μmmol) of Pd(PPh₃)₂Cl₂ and the reaction mixture was heated under reflux for 5 hours under a nitrogen atmosphere. After allowing the reaction mixture to cool to room temperature, 30 ml of ethyl acetate and 30 ml of saturated aqueous ammonium chloride were added and layers were separated. The aqueous layer was extracted twice with 30 ml of ethyl acetate. The organic layers were combined, dried over magnesium sulfate and concentrated under reduced pressure. The residue was purified by silica column chromatography (n-hexane:ethyl acetate=15:1) to obtain 5.7 g (yield, 83.1%; HPLC purity, 97.5%) of the desired compound.

Compound (B-3) ESI-MS (m/z) m/z=277.1 [M+H]+

### Synthesis of Compound (B-4)

To a 200 ml two-necked flask, there were charged 100 ml of 30% HBr-AcOH and 5.5 g (19.9 mmol) of compound (B-3), and the reaction mixture was heated under reflux for 10 hours. After distilling off the solvent by concentration under ordinary pressure, 100 ml of 30% HBr-AcOH was again added and the mixture was heated under reflux for 10 hours. The reaction mixture was concentrated under ordinary pressure and allowed to cool to room temperature. Thereafter, 50 ml of ethanol and 5 ml of 98% sulfuric acid were added thereto and the reaction mixture was heated under reflux for 8 hours. The reaction mixture was concentrated under reduced pressure and the residue was dissolved by adding 50 ml of ethanol. This solution was added dropwise to 30 ml of 10% aqueous sodium hydroxide and the pH was adjusted to 8 to 9 with 10% aqueous sodium hydroxide. After separating the organic layer and aqueous layer, the aqueous layer was extracted twice with 30 ml of ethyl acetate. The organic layers were combined, dried over magnesium sulfate and concentrated under reduced pressure. The residue was purified by silica column chromatography (n-hexane:ethyl acetate=10:1) to obtain 5.7 g (yield, 89%; HPLC purity, 55.5%) of the desired compound.

Compound (B-4) ESI-MS (m/z) m/z=321.0 [M+H]+

### Synthesis of Compound (B-7)

To a 300 ml four-necked flask, there were charged successively 100 ml of DMF, 1.7 g (5.3 mmol) of compound (B-4) and 0.87 g (6.4 mmol) of p-methoxybenzaldehyde. To this was added 1.3 g (11.6 mmol) of t-BuOK and the reaction mixture was stirred at room temperature for 13 hours under a nitrogen atmosphere. The solvent was distilled off by concentration under reduced pressure, followed by addition of 50 ml of ethyl acetate and 50 ml of deionized water. The pH was adjusted to a range of 6 to 7 with 2N hydrochloric acid. After separating the organic layer and aqueous layer, the aqueous layer was extracted twice with 50 ml of ethyl acetate. The organic layers were combined, dried over magnesium sulfate and concentrated under reduced pressure. Thereafter, 70 ml of ethanol and 7 ml of 98% sulfuric acid were added and the mixture was heated under reflux for 8 hours under a nitrogen atmosphere. The solvent was distilled off by concentration under reduced pressure. Thereafter, 50 ml of ethyl acetate was added and the residue was dissolved. This solution was added dropwise to 20 ml of 10% aqueous sodium hydroxide and the pH was adjusted to a range of 8 to 9 with 10% aqueous sodium hydroxide. After separating the organic layer and aqueous layer, the aqueous layer was extracted twice with 50 ml of ethyl acetate. The organic layers were combined, dried over magnesium sulfate and concentrated under vacuum. The residue was purified by silica column chromatography (n-hexane:ethyl acetate=8:1) to obtain 1.8 g (yield, 77%; HPLC purity, 90.4%) of the desired compound.

Compound (B-7) ESI-MS (m/z) m/z=439.1 [M+H]+

### Synthesis of Compound (B-8)

To a 50 ml two-necked flask, there were added 30 ml of DME, 350 mg (0.80 mmol) of compound (B-7), 783 mg (2.4 mmol) of Me₃Sn—SnMe₃ and 27.0 mg (23.4 μmol) of Pd(PPh₃)₄, and the mixture was heated under reflux for 6 hours under a nitrogen atmosphere. After allowing the reaction mixture to cool to room temperature, the solvent was distilled off by concentration under reduced pressure, whereupon 50 ml of diethyl ether was added and the mixture was stirred at room temperature for 12 hours. The solution was filtered and the filtrate was concentrated under reduced pressure to proceed to the next process.

Compound (B-8) ESI-MS (m/z) m/z=525.1 [M+H]+

### Synthesis of Compound (B-9)

To a 50 ml two-necked flask were added 30 ml of DME, compound (B-8) synthesized in the previous process, 385 mg (0.88 mmol) of compound (B-7) and 39 mg (55.6 μmol) of Pd(PPh₃)₂Cl₂, and the mixture was heated under reflux for 6 hours under a nitrogen atmosphere.

After the reaction mixture was cooled gradually to 10° C., it was stirred for 12 hours at inner temperature in a range of 10 to 15° C., filtered, and washed to obtain 367 mg (yield, 64% (yield based on compound (B-7); HPLC purity, 87.7%) of the desired material.

Compound (B-9) ESI-MS (m/z) m/z=719.3 [M+H]+

### Synthesis of Compound (I′-37)

To a 50 ml two-necked flask, there were charged 20 ml of ethanol, 22 mg (0.031 mmol) of compound (B-9), 3.7 mg (0.015 mmol) of LiOH and 5 ml of deionized water, and the reaction mixture was heated under reflux for 10 hours. After adjusting the pH to 6 to 7 with 2N hydrochloric acid, the reaction mixture was concentrated under reduced pressure. To the residue were added 20 ml of DMF and 17.0 mg (0.082 mmol) of RuCl₃, and the mixture was stirred at a temperature range of 110 to 120° C. for 10 hours under a nitrogen atmosphere. To this reaction mass was added a solution of 44 mg (0.58 mmol) of NH₄SCN dissolved in 5 ml of deionized water and heating was continued for 10 more hours. After being allowed to cool to room temperature, the reaction mixture was concentrated under vacuum, and the main component of the residue was fractionated by high-performance liquid chromatography to obtain a solid material.

Compound (I′-37) ESI-MS (m/z) m/z=880.0 [M]+

### <Preparation of a Photoelectrochemical Cell Comprising Compound (I′-37)>

On a conductive surface of a conductive substrate, conductive glass provided with a tin oxide film doped with fluorine (manufactured by Nippon Sheet Glass Co., Ltd., 10 Ω/sq), a dispersion liquid of titanium oxide, Ti-Nanoxide T/SP (trade name, manufactured by Solaronix SA) was coated by means of a screen printer and, thereafter burned at 500° C. The glass was cooled to obtain a layer of semiconductor particles laminated on a conductive substrate. Subsequently, the glass was dipped for 16 hours in a solution of compound (1-37) (the concentration, 0.0003 mol/l; solvent, N,N-dimethylacetamide/ethanol (1:1 (v/v)); 0.40 mol/l of chenodeoxycholic acid was added). After taking the glass out of the solution, it was washed with acetonitrile and dried naturally to obtain a laminated body (the area of the titanium oxide electrode was 24 mm²) comprising a conductive substrate and a layer of semiconductor fine particles on which photosensitizing dyes are adsorbed. Then, after disposing a 25 μm-thick polyethylene terephthalate film around the layer as a spacer, the layer was impregnated with an electrolytic solution (solvent, acetonitrile; iodine concentration in the solvent, 0.05 mol/l; lithium iodide concentration in the solvent, 0.1 mol/l; 4-t-butylpyridine concentration in the solvent, 0.5 mol/l; 1-propyl-2,3-dimethylimidazolium iodide concentration in the solvent, 0.6 mol/l). Finally, glass vapor-deposited with platinum, which is the counter electrode, was superposed to obtain a photoelectrochemical cell, wherein a conductive substrate, a layer of semiconductor fine particles on which photosensitizing dyes are adsorbed, and a counter electrode of the conductive substrate are laminated with an electrolytic solution impregnated between the conductive substrate and the counter electrode. With thus prepared photoelectrochemical cell, using an IPCE (incident photon-to-current efficiency) measuring instrument (manufactured by Bunko Keiki Co., Ltd), the IPCE was measured. The results are shown in Table 7.

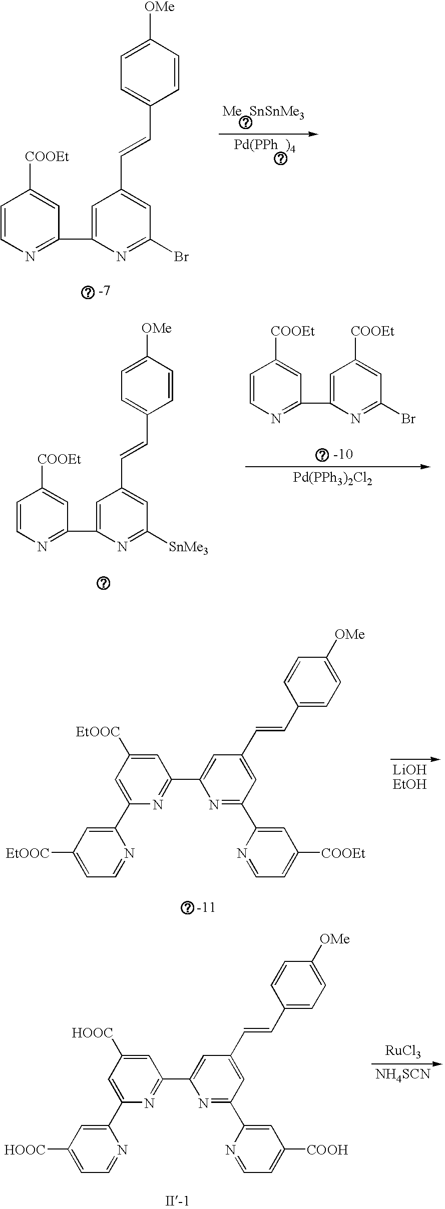

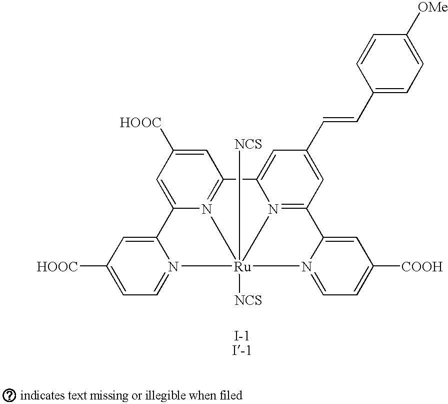

### Example 5

### Manufacturing Example 5

### Manufacturing Example of Compound (I′-1)

### Synthesis of (B-11)

According to the method described in Example 1, compound (B-8) was synthesized from 320 mg (0.73 mmol) of compound (B-7). To a two-necked flask were added 30 ml of DME, compound (B-8) synthesized, 307 mg (0.81 mmol) of compound (B-10) and 20 mg (28.5 μmol) of Pd(PPh₃)₂Cl₂, and the mixture was heated under reflux for 6 hours under a nitrogen atmosphere. The reaction mixture was cooled gradually to 10° C., stirred at inner temperature in a range of 10 to 15° C. for 12 hours, filtered, and washed to obtain 216 mg (yield, 45% (yield based on compound (B-7)); HPLC purity, 88.9%) of the desired material.

Compound (B-11) ESI-MS (m/z) m/z=659.2 [M+H]+

### Synthesis of Compound (I′-1)

To a 50 ml two-necked flask, there were charged 20 ml of ethanol, 215 mg (0.33 mmol) of compound (B-11), 40.3 mg (1.68 mmol) of LiOH and 5 ml of deionized water, and the reaction mixture was heated under reflux for 10 hours. The pH was adjusted to 6 to 7 with 2N hydrochloric acid and the reaction mixture was concentrated under reduced pressure. To the residue was added 20 ml of DMF and 82.2 mg (0.40 mmol) of RuCl₃, and the mixture was stirred at a temperature range of 110 to 120° C. for 10 hours under a nitrogen atmosphere. To this reaction mass was added a solution of 378.2 mg (4.97 mmol) of NH₄SCN dissolved in 10 ml of deionized water and heating was continued for further 10 hours. After the reaction mixture was allowed to cool to room temperature, it was concentrated under reduced pressure, and the main component of the residue was fractionally collected by high-speed liquid chromatography to obtain a solid material.

Compound (I′-1) ESI-MS (m/z) m/z=792.2 [M]+

Except that THF was used as the solvent and chenodeoxycholic acid was added in an amount of 0.10 mol/l, the IPCE was measured in the same manner as in Example 4. The IPCE of the photoelectric converter obtained in Example 5 is shown in Table 7.

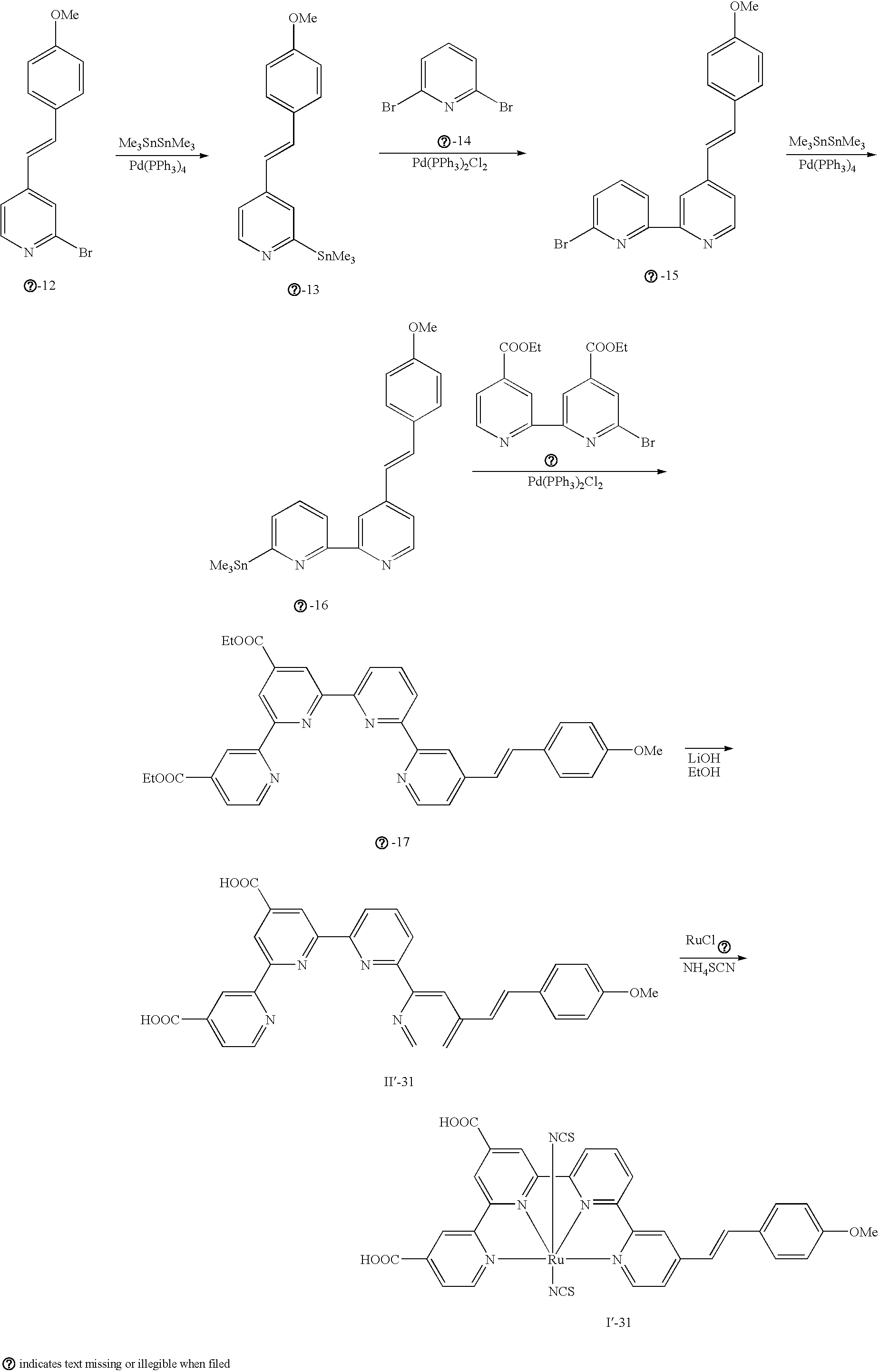

### Example 6

### Manufacturing Example 6

### Manufacturing Example of Compound (I′-31)

### Synthesis of Compound (B-12)

To a 300 ml four-necked flask, there were charged successively 100 ml of DMF, 20.0 g (0.12 mol) of 2-bromopicoline and 15.8 g (0.12 ml) of p-methoxybenzaldehyde. To this was added 16.2 g (0.15 mol) of t-BuOK and the reaction mixture was stirred at room temperature for 13 hours under a nitrogen atmosphere. The solvent was distilled off by concentration under reduced pressure and, thereafter, 100 ml of ethyl acetate and 100 ml of deionized water were added. The pH was adjusted to a range of 6 to 7 with 2N hydrochloric acid. After separating an organic layer and an aqueous layer, the aqueous layer was extracted twice with 100 ml of ethyl acetate. The organic layers were combined, dried over magnesium sulfate and concentrated under reduced pressure. The residue was purified by silica column chromatography (n-hexane:ethyl acetate=10:1→1:1→1:5) to obtain 11.5 g (yield, 69%; HPLC purity, 90.1%) of the desired compound.

Compound (B-12) ESI-MS (m/z) m/z=290.0 [M+H]+

### Synthesis of Compound (B-13)

Synthesis was carried out in the same manner as in the synthesis of compound (B-8) described in Example 1, except that compound (B-7) was replaced with (B-12).

Compound (B-13) ESI-MS (m/z) m/z=376.1 [M+H]+

### Synthesis of Compound (B-15)

To a 200 ml two-necked flask were added 50 ml of toluene, compound (B-13) synthesized from 290 mg (1.00 mmol) of compound (B-12) in the previous process, 353.4 mg (1.49 mmol) of compound (B-14) and 78.6 mg (112.0 μmol) of Pd(PPh₃)₂Cl₂ and the reaction mixture was stirred under heating for 2 hours at inner temperature in a range of 100° C. to 105° C. under a nitrogen atmosphere. After cooling to room temperature, the reaction mixture was concentrated and purified by silica column chromatography (n-hexane:ethyl acetate=10:1→2:1) to obtain 186.4 mg (yield, 89%; HPLC purity, 55.5%) of the desired compound. The desired compound was obtained (yield, 51% (yield based on compound (B-12); HPLC purity, 98.7%).

Compound (B-15) ESI-MS (m/z) m/z=367.1 [M+H]+

### Synthesis of Compound (B-16)

Synthesis was carried out in the same manner as in the synthesis of compound (B-8) described in Example 1 except that compound (B-7) was replaced with compound (B-15).

Compound (B-16) ESI-MS (m/z) m/z=452.1 [M+H]+

### Synthesis of Compound (B-17)

Synthesis was carried out in the same manner as in the synthesis of compound (B-15) described in Example 3, except that compound (B-13) was replaced with compound (B-16) and compound (B-14) was replaced with compound (B-10), respectively.

Compound (B-17) ESI-MS (m/z) m/z=587.2 [M+H]+

### Synthesis of (I′-31)

Synthesis was carried out in the manner as the synthesis of compound (I′-37) described in Example 4, except that compound (B-9) was replaced with (B-17).

Compound (I′-31) ESI-MS (m/z) m/z=748.0 [M]+

Except that chenodeoxycholic acid was added in an amount of 0.16 mol/l, the IPCE was measured in the same manner as in Example 4. The IPCE of the photoelectric converter obtained in Example 6 is shown in Table 7.

### Comparative Example 3

A photoelectrochemical cell was obtained in the same manner as in Example 4 except that cis-bis(isothiocyanato)bis(2,2′-bipyridyl-4,4′-dicarboxylate)-ruthenium (compound (1)) was used as a photosensitizing dye, and ethanol was used as a dissolving solvent. Then, the IPCE was measured in the same manner as in Example 4. The results are summarized in Table 7.

TABLE 7

| | | | | Comparative |
|---|---|---|---|---|
| | Example 4 | Example 5 | Example 6 | Example 3 |
| Compound | I′-37 | I′-1 | I′-31 | (1) |
| IPCE (750 nm) | 9.22% | 37.19% | 19.70% | 5.14% |
| IPCE (800 nm) | 3.65% | 23.23% | 10.58% | 0.29% |
| IPCE (850 nm) | 0.72% | 11.79% | 3.55% | 0.03% |
| IPCE (900 nm) | 0.41% | 2.20% | 0.51% | 0.02% |

### INDUSTRIAL APPLICABILITY

The complex compound of the present invention has excellent photoelectric conversion efficiency not only in a visible light region but also in a long-wavelength region of 750 nm or longer, and can be suitably used as a photosensitizing dye. In addition, a photoelectric converter comprising the complex compound has a high photoelectric conversion efficiency and can be used for a solar cell utilizing sun light and for a photoelectrochemical cell utilizing artificial light found in a tunnel or inside a house. Also, because the photoelectric converter generates an electric current by irradiation of light, it can also be used as an optical sensor.

1. A complex compound (I), wherein a compound represented by the following formula (II), abbreviated as compound (II), is coordinated to a metal atom: 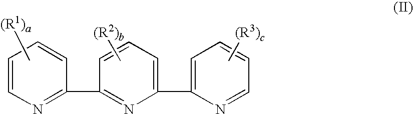
wherein, R¹, R² and R³ each independently represent a substituent represented by the following formula (III), formula (IV), formula (V) or formula (VI), and at least one of these is a substituent represented by the formula (III); a, b and c each independently represent 0 or an integer of 1 to 2 and a+b+c≧1:
-L-Ar-A (III)
-L-Ar—Y (IV)
-A (V)
—Y (VI)
wherein L represents a linking group represented by the following formula (VII) or formula (VIII); Ar represents an aryl group which may have a substituent; A represents an acidic group or a salt thereof; Q¹ and Q² each independently represent a hydrogen atom, an alkyl group having 1 to 20 carbon atoms, an alkoxy group having 1 to 20 carbon atoms, an aryl group having 6 to 20 carbon atoms or a cyano group; p and q each represent an integer of 1 to 3: 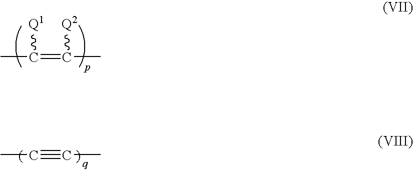
and Y represents at least one group selected from the group consisting of a hydrogen atom, an alkyl group having 1 to 20 carbon atoms, an alkoxy group having 1 to 20 carbon atoms, an alkoxyalkyl group having 2 to 20 carbon atoms, an aryloxy group having 6 to 20 carbon atoms, an arylalkyloxy group having 7 to 20 carbon atoms, an aryloxyalkyl group having 7 to 20 carbon atoms, an alkylthio group having 1 to 20 carbon atoms, an alkylthioalkyl group having 2 to 20 carbon atoms, an arylthio group having 6 to 20 carbon atoms, an arylalkylthio group having 7 to 20 carbon atoms, an arylthioalkyl group having 7 to 20 carbon atoms, an alkylsulfonyl group having 1 to 20 carbon atoms, an arylsulfonyl group having 6 to 20 carbon atoms, an amino group containing two of an alkyl group having 1 to 20 carbon atoms or an aryl group having 6 to 20 carbon atoms and a cyano group.

2. The complex compound (I) according to claim 1, wherein L in the formula (III) comprises a substituent represented by the formula (VII), wherein, R¹, R², R³, a, b, c, A, L, Ar, Y, p, Q¹ and Q² represent the same meanings as in claim 1.

3. The complex compound (I) according to claim 1, wherein L in the fowl:lila (III) comprises a substituent represented by the formula (VIII), wherein, R¹, R², R³, a, b, c, A, L, Ar, Y and q represent the same meanings as in claim 1.

4. The complex compound (I) according to claim 1, wherein the acidic group is at least one group selected from the group consisting of a carboxyl group, a sulfonic acid group, a squaric acid group, a phosphoric acid group and a boric acid group.

5. The complex compound (I) according to claim 4, wherein the acidic group is a carboxylic group.

6. The complex compound (I) according to claim 1, wherein the salt of the acidic group is a salt with an organic base.

7. The complex compound (I) according to claim 1, wherein at least one of R¹, R² and R³ is a substituent represented by the formula (III) according to claim 1; the linking group L is represented by the formula (VII); Q¹ and Q² are hydrogen atoms; p is 1; Ar is a thiophene ring which may have a substituent; and A is a carboxyl group.

8. The complex compound (I) according to claim 1, wherein a+b+c is an integer of 1 to 3.

9. The complex compound (I) according to claim 1, wherein the metal atom is Fe, Ru or Os.

10. A compound represented by the formula (II): 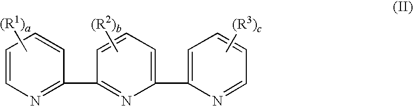
wherein, R¹, R², R³, a, b, c, A, L, Ar, Y, p, q, Q¹ and Q² represent the same meanings as in claim 1.

11. The compound (II) represented by the formula (II) according to claim 10, wherein L in the formula (III) is a compound comprising a substituent represented by the formula (VII).

12. The compound (II) represented by the formula (II) according to claim 10, wherein L in the formula (III) is a compound comprising a substituent represented by the formula (VIII).

13. The compound (II) according to claim 10, wherein the acidic group is at least one group selected from the group consisting of a carboxyl group, a sulfonic acid group, a squaric acid group, a phosphoric acid group and a boric acid group.

14. The compound (II) according to claim 13, wherein the acidic group is a carboxyl group.

15. The compound (II) according to claim 10, wherein the salt of the acidic group is a salt with an organic base.

16. The compound (II) according to claim 10, wherein at least one of R¹, R² and R³ is a substituent represented by the formula (III) according to claim 1; the linking group L is represented by the formula (VII); Q¹ and Q² are hydrogen atoms and p is 1; Ar is a thiophene ring which may have a substituent; and A is a carboxyl group.

17. The compound (II) according to claim 10, wherein a+b+c is an integer of 1 to 3.

18. A photosensitizing dye comprising a complex compound (I) according to claim 1.

19. A photoelectric converter comprising a conductive substrate and a layer of semiconductor fine particles on which a photosensitizing dye according to claim 18 is adsorbed.

20. A photoelectrochemical cell comprising a photoelectric converter according to claim 19, a charge transport layer and a counter electrode.

21. A method for manufacturing a tin compound represented by the following formula (A) or (B), wherein a halogenated compound (IX) or (X) described in the following formula is reacted with a tin reagent represented by the following formula (XI) in the presence of a metal catalyst: 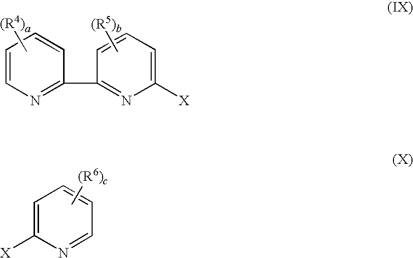
a tin reagent represented by the formula (XI): 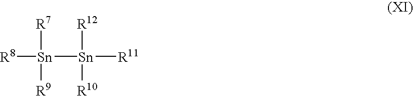
the following (A) and (B) represent reaction products of (IX) or (X) with (XI): 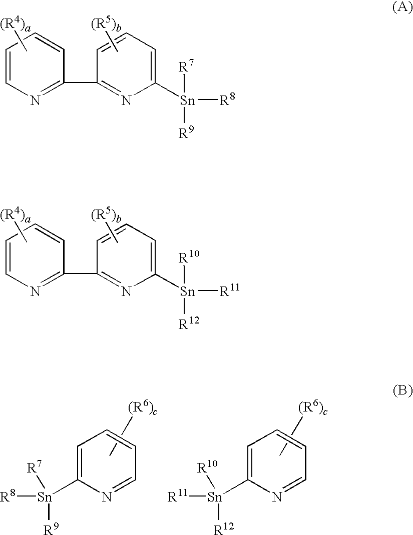
wherein, R⁴, R⁵ and R⁶ each independently represent a substituent represented by the formula (XII), formula (XIII), formula (XIV) or formula (XV), and at least one of these is a substituent represented by the formula (XII); a, b and c represent the same meanings as in claim 1; R⁷, R⁸, R⁹, R¹⁰, R¹¹ and R¹² each independently represent an alkyl group having 1 to 6 carbon atoms; X represents a halogen atom;
-L-Ar—B (XII)
-L-Ar—Y (XIII)
—B (XIV)
—Y (XV)
wherein, L, Ar and Y represent the same meanings as in claim 1; B represents an acidic group to which a protecting group is introduced.

22. A method for manufacturing the compound represented by the (II) 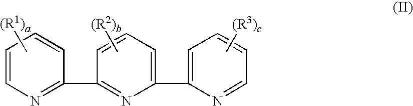
wherein, R¹, R² and R³ each independently represent a substituent represented by the following formula (III), formula (IV), formula (V) or formula (VI), and at least one of these is a substituent represented by the formula (III); a, b and c each independently represent 0 or an integer of 1 to 2 and a+b+c≧1:
-L-Ar-A (III)
-L-Ar—Y (IV)
-A (V)
—Y (VI)
wherein L represents a linking group represented by the following formula (VII) or formula (VIII); Ar represents an aryl group which may have a substituent; A represents an acidic group or a salt thereof; Q¹ and Q² each independently represent a hydrogen atom, an alkyl group having 1 to 20 carbon atoms, an alkoxy group having 1 to 20 carbon atoms, an aryl group having 6 to 20 carbon atoms or a cyano group; p and q each represent an integer of 1 to 3: 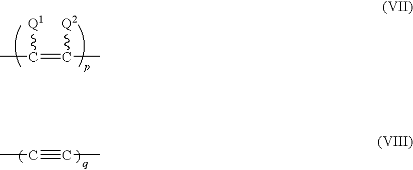
and Y represents at least one group selected from the group consisting of a hydrogen atom, an alkyl group having 1 to 20 carbon atoms, an alkoxy group having 1 to 20 carbon atoms, an alkoxyalkyl group having 2 to 20 carbon atoms, an aryloxy group having 6 to 20 carbon atoms, an arylalkyoxy having 7 to 20 carbon atoms, an aryloxyalkyl group having 7 to 20 carbon atoms, an alkylthio group having 1 to 20 carbon atoms, an alkylthioalkyl group having 2 to 20 carbon atoms, an arylthio group having 6 to 20 carbon atoms, an arylalkylthio group having 7 to 20 carbon atoms, an arylthioalkyl group having 7 to 20 carbon atoms, an alkylsulfonyl group having 1 to 20 carbon atoms, an arylsulfonyl group having 6 to 20 carbon atoms, an amino group containing two of an alkyl group having 1 to 20 carbon atoms or an aryl group having 6 to 20 carbon atoms and a cyano group,
wherein a tin compound represented by the formula (A) obtained by the manufacturing method according to claim 21 and a halogenated compound (X) are, or a tin compound represented by the formula (B) and a halogenated compound (IX) are subjected to a coupling reaction in the presence of a metal catalyst to obtain the following compound (XVI), followed by hydrolysis of the compound (XVI): 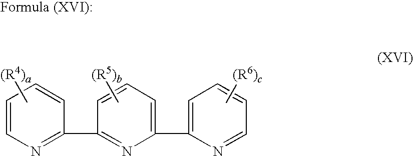
wherein R⁴, R⁵, R⁶, a, b and c represent the same meanings as in claim 21.

23. A method for manufacturing the compound (II), 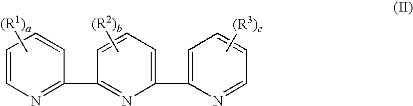
wherein, R¹, R² and R³ each independently represent a substituent represented by the following formula (III), formula (IV), formula (V) or formula (VI), and at least one of these is a substituent represented by the formula (III); a, b and c each independently represent 0 or an integer of 1 to 2 and a+b+c≧1:
-L-Ar-A (III)
-L-Ar—Y (IV)
-A (V)
—Y (VI)
wherein L represents a linking group represented by the following formula (VII) or formula (VIII); Ar represents an aryl group which may have a substituent; A represents an acidic group or a salt thereof: Q¹ and Q² each independently represent a hydrogen atom, an alkyl group having 1 to 20 carbon atoms, an alkoxy group having 1 to 20 carbon atoms, an aryl group having 6 to 20 carbon atoms or a cyano group; p and q each represent an integer of 1 to 3: 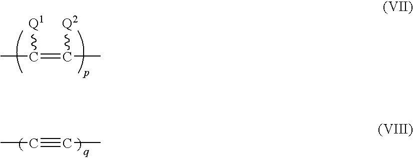
and Y represents at least one group selected from the group consisting of a hydrogen atom, an alkyl group having 1 to 20 carbon atoms, an alkoxy group having 1 to 20 carbon atoms, an alkoxyalkyl group having 2 to 20 carbon atoms, an aryloxy group having 6 to 20 carbon atoms, an arylalkyloxy group having 7 to 20 carbon atoms, an aryloxyalkyl group having 7 to 20 carbon atoms, an alkylthio group having 1 to 20 carbon atoms, an alkylthioalkyl group having 2 to 20 carbon atoms, an arylthio group having 6 to 20 carbon atoms, an arylalkylthio group having 7 to 20 carbon atoms, an arylthioalkyl group having 7 to 20 carbon atoms, an alkylsulfonyl group having 1 to 20 carbon atoms, an arylsulfonyl group having 6 to 20 carbon atoms, an amino group containing two of an alkyl group having 1 to 20 carbon atoms or an aryl group having 6 to 20 carbon atoms and a cyano group,
wherein the tin compound represented by the formula (B) manufactured from the halogenated compound (X) by using the manufacturing method according to claim 21 is subjected to a coupling reaction with a halogenated compound represented by the following formula (XVII) in the presence of a metal catalyst to obtain the compound (XVI) 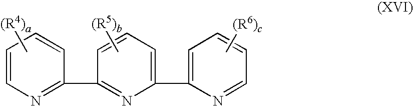
R⁴, R⁵, R⁶, a, b and c represent the same meanings as in claim 21, followed by hydrolysis thereof: 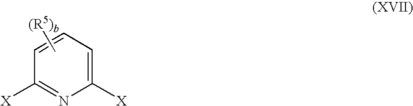
R⁵, b and X represent the same meanings as in claim 21 and (R⁴)ₐ═(R⁶)_{c}.

24. A complex compound (I′), wherein a compound represented by the following formula (II′), abbreviated as compound (II′), is coordinated to a metal atom: 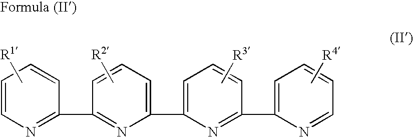
wherein, R¹′, R²′, R³′ and R⁴′ are each independent, at least one of R¹′ to R⁴′ is an acidic group or a salt thereof, at least one of them is represented by the formula (III′): 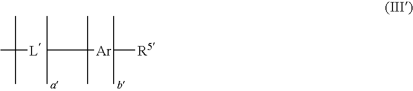
and at least one of them is represented by the formula (III′) where a′=1; wherein a′ and b′ are each independent and represent an integer of 0 or 1; R¹′ to R⁵′ represent an acidic group or a salt thereof, a hydrogen atom, or a substituent; herein the substituent is a group selected from the group consisting of an alkyl group having 1 to 20 carbon atoms, an alkoxy group having 1 to 20 carbon atoms, an alkoxyalkyl group having 2 to 20 carbon atoms, an aryloxy group having 6 to 20 carbon atoms, an arylalkyloxy group having 7 to 20 carbon atoms, an aryloxyalkyl group having 7 to 20 carbon atoms, an ester group having 2 to 20 carbon atoms, an alkylthio group having 1 to 20 carbon atoms, an alkylthioalkyl group having 2 to 20 carbon atoms, an arylthio group having 6 to 20 carbon atoms, an arylalkylthio group having 7 to 20 carbon atoms, an arylthioalkyl group having 7 to 20 carbon atoms, an alkylsulfonyl group having 1 to 20 carbon atoms, an arylsulfonyl group having 6 to 20 carbon atoms, an amino group containing two of an alkyl group having 1 to 20 carbon atoms or an aryl group having 6 to 20 carbon atoms and a cyano group; Ar represents an aryl group which may have a substituent; and L′ is a group represented by the following formula (IV′): 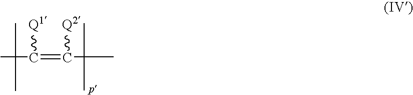
or the following formula (V′): 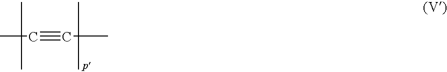
wherein, Q¹′ and Q²′ each independently represent a hydrogen atom, an alkyl group having 1 to 20 carbon atoms, an alkoxy group having 1 to 20 carbon atoms, an aryl group having 6 to 20 carbon atoms or a cyano group; and p′ is an integer of 1 to 3.

25. The complex compound (I′) according to claim 24, wherein the acidic group is a group selected from the group consisting of a carboxyl group, a sulfonic acid group, a squaric acid group, a phosphoric acid group and a boric acid group.

26. The complex compound (I′) according to claim 25, wherein the acidic group is a carboxyl group.

27. The complex compound (I′) according to claim 24, wherein the salt of the acidic group is a salt with an organic base.

28. The complex compound (I′) according to claim 24, wherein the number of the acidic groups or the salts thereof are two or more.

29. The complex compound (I′) according to claim 24, wherein, in the formula (III′), b′=1 and R⁵′ is an alkoxy group.

30. The complex compound (I) according to claim 24, wherein, in the formula (III′), b′=1 and R⁵′ is a carboxyl group or a salt thereof.

31. The complex compound (I) according to claim 24, wherein, in the formula (III′), L′ represents the formula (IV′); Q¹′ and Q²′ represent hydrogen atoms; p′=1 and b′=1; and Ar represents a benzene ring or a benzene ring having a substituent.

32. The complex compound (I′) according to claim 24, wherein, in the formula (III), L′ represents the formula (IV′); Q¹′ and Q²′ represent hydrogen atoms; p′=1 and b′=1; and Ar represents a thiophene ring or a thiophene ring having a substituent.

33. A compound represented by the formula (II′): 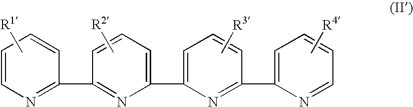
wherein, R¹′, R²′, R³′, R⁴′, R⁵′, Q¹′, Q²′, Ar, L′, a′, b′ and p′ represent the same meanings as in claim 24.

34. The compound (II′) according to claim 33, wherein the acidic group is a group selected from the group consisting of a carboxyl group, a sulfonic acid group, a squaric acid group, a phosphoric acid group and a boric acid group.

35. The compound (II′) according to claim 34, wherein the acidic group is a carboxyl group.

36. The compound (II′) according to claim 33, wherein the salt of the acidic group is a salt with an organic base.

37. The compound (II′) according to claim 33, wherein the number of the acidic groups or the salts thereof are two or more.

38. The compound (II′) according to claim 33, wherein, in the formula (III′), b′=1 and R⁵′ is an alkoxy group.

39. The compound (II′) according to claim 33, wherein, in the formula (III′), b′=1 and R⁵′ is a carboxyl group or a salt thereof.

40. The compound (II′) according to claim 33, wherein, in the formula (III′), L′ represents the formula (IV); Q¹′ and Q²′ are hydrogen atoms; p′=1 and b′1; and Ar is a benzene ring or a benzene ring having a substituent.

41. The complex compound (II′) according to claim 33, wherein, in the formula (III′), L′ represents the formula (IV′); Q1′ and Q2′ are hydrogen atoms; p′=1 and b′=1; and Ar is a thiophene ring or a thiophene ring having a substituent.

42. A method for manufacturing the compound (II′) according to claim 33, comprising the following processes (A) to (C) or processes (A) and (B):
[Process A]:
a process wherein a compound represented by the formula (1% hereinafter abbreviated as compound (1): 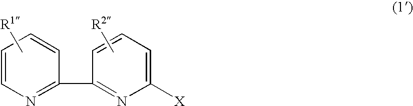
wherein, X represents a halogen atom;
and a compound represented by the formula (2′), hereinafter abbreviated as compound (2′):
(⁶′R)₃—Sn—Sn—(R⁶′)₃ (2′)
wherein, R⁶″ represents an alkyl group having 1 to 4 carbon atoms;
are reacted to obtain a compound represented by the formula (3′), hereinafter abbreviated as compound (3′): 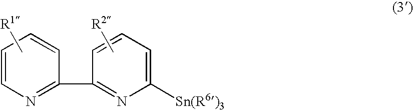
[Process B]:
a process wherein the compound (3′) obtained in the process (A) and a compound represented by the formula (4′), hereinafter abbreviated as compound (4′): 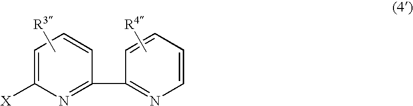
wherein, X represents a halogen atom;
are reacted in the presence of a metal catalyst to obtain a compound represented by the formula (5′), hereinafter abbreviated as compound (5′): 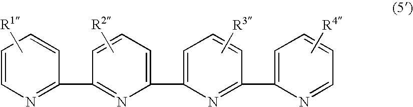
wherein, R¹″, R²″, R³″ and R⁴″ are each independent, at least one of R¹″ to R⁴″ is an acidic group to which a protecting group is introduced, and at least one of them is represented by the formula (IX): 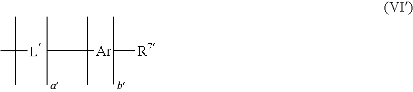
and at least one of them is a group represented by the formula (VI′) where a′=1; wherein in the formula (VI′), a′, b′, Ar and L′ represent the same meanings as the definitions described in the formula (III′); R¹′ to R⁴′ and R⁷′ represent an acidic group to which a protecting group is introduced, a hydrogen atom or a substituent; and the substituent represents the same meaning as the definition described in the formula (III').
[Process C]:
a process wherein the compound (II′) is obtained by removing in a solvent the protecting group of compound (5′) obtained in the process (B).

43. The method for manufacturing the compound (II') according to claim 42, wherein X is a bromine atom, R⁶′ is a methyl group or an n-butyl group and the protected acidic group is a methyl ester or an ethyl ester.

44. The method for manufacturing the compound (II′) according to claim 42, wherein the metal catalyst is Pd(PPh₃)₄ or Pd(PPh₃)₂Cl₂.

45. The method for manufacturing the compound (II′) according to claim 42, wherein a base is used in removing the protecting group which has been introduced, the base is lithium hydroxide or triethylamine, and the solvent is methanol or ethanol.

46. The complex compound (I′) according to claim 24, wherein the metal atom is Fe, Ru or Os.

47. A photosensitizing dye, comprising the complex compound (I′) according to claim 24.

48. A photoelectric converter comprising a conductive substrate and a layer of semiconductor fine particles on which the photosensitizing dye according to claim 47 is adsorbed.

49. A photoelectrochemical cell comprising the photoelectric converter according claim 48, a charge transport layer and a counter electrode.
